(19) United States
(12) Patent Application Publication
(10) Pub. No.: US 2005/0019366 A1
(43) Pub. Date: Jan. 27, 2005

(54) Drug-coated stents and methods of use therefor

(76) Inventor: Jerome B. Zeldis, Princeton, NJ (US)

Correspondence Address: JONES DAY, 222 EAST 41ST ST, NEW YORK, NY, 10017, US

(21) Appl. No.: 10/749,344

(22) Filed: Dec. 30, 2003

Related U.S. Application Data
(60) Provisional application No. 60/437,332, filed on Dec. 31, 2002.

(51) Int. Cl. A61K 31/415; A61F 2/00

(52) U.S. Cl. 424/423; 514/406

(57) ABSTRACT

This invention relates to stents comprising an effective amount of a c-Jun N-terminal kinase (“JNK”) Inhibitor, the stents being useful for treating or preventing a cardiovascular or renal disease. The invention also relates to the treatment or prevention of cardiovascular or renal disease, such as atherosclerosis or restenosis, comprising implanting into a patient in need thereof of a stent comprising an effective amount of a JNK Inhibitor.

This application claims the benefit of U.S. provisional application No. 60/437,332, filed Dec. 31, 2002, the contents of which are incorporated by reference herein in their entirety.

### 1. FIELD OF INVENTION

This invention relates to stents comprising an effective amount of a c-Jun N-terminal kinase (“JNK”) Inhibitor, the stents being useful for treating or preventing a cardiovascular or renal disease. The invention also relates to the treatment or prevention of cardiovascular or renal disease, such as atherosclerosis or restenosis, comprising implanting into a patient in need thereof of a stent comprising an effective amount of a JNK Inhibitor.

### 2. BACKGROUND OF THE INVENTION

2.1 Pathobiology of Atherosclerosis and Restinosis

In 1999, there were almost 1 million deaths due to vascular disease in the United States (twice as many as from cancer and 10 times as many as from accidents). National Vital Statistics Reports, Vol. 49, No. 8. Vascular disease may affect the brain, heart, kidneys and other vital organs as well as the extremities. Ross R., Annu. Rev. Physiol. 57:791-804, 1995.

The most common and serious vascular disease is atherosclerosis. Atherosclerosis is characterized by patchy subintimal thickening (atheromas) of the arteries and involves the whole arterial vessel tree. Espinola-Klein C. et al., Med. Klin. 97(4):221-228, 2002.

Development of atherosclerotic lesions involves proliferation of cellular constituents of the wall of blood vessels in response to chemical stimuli from platelets and monocytes derived from the blood. This proliferation of cells in the vessel wall can lead to narrowing of the lumen of the vessel. In addition, atherosclerotic plaques, the focal lesions of atherosclerosis, can be sites of thrombus or clot formation, hemorrhage, or ulceration leading to interruption of the blood supply of the organ supplied by the affected blood vessel.

Two main hypotheses have been proposed to explain the pathogenesis of atherosclerosis: the lipid hypothesis and the chronic endothelial injury hypothesis.

The lipid hypothesis postulates that an elevation in plasma LDL levels results in penetration of LDL into the arterial wall, leading to lipid accumulation in smooth muscle cells and in macrophages (foam cells).

The chronic endothelial injury hypothesis postulates that endothelial injury by various mechanisms produces loss of endothelium, adhesion of platelets to subendothelium, aggregation of platelets to subendothelium, aggregation of platelets, chemotaxis of monocytes and T-cell lymphocytes, and release of the platelet-derived and monocyte-derived growth factors that induce migration of smooth muscle cells from the media into the intima, where they replicate, synthesize connective tissue and proteoglycans and form a fibrous plaque.

Further, it has been suggested that the level of macrophage colony stimulating factor (M-CSF) in the atherectomy tissue can indicate, or predict the likelihood or degree of, restenosis in post vascular intervention tissue. Takano et al., Circulation, 98 (17, Supp): 4437, 1998.

Atherosclerosis is characteristically asymptomatic until critical stenosis, thrombosis, aneurysm or embolus supervenes. Initially, symptoms and signs reflect an inability of blood flow to the affected tissue to increase with demand (e.g., angina or exertion, intermittent claudication). Symptoms and signs commonly develop gradually as the atheroma slowly encroach on the vessel lumen. However, when a major artery is acutely occluded, the results can be serious, such as, for example, infarction of heart muscle as described above.

Traditional therapy for prevention or inhibition of cardiovascular and cerebrovascular complications of atherosclerosis is an indirect approach aimed at reducing or reversing the risk factors associated with atherosclerosis such as cigarette smoking, obesity, abnormal serum levels (LDL cholesterol levels), hypertension, diabetes mellitus, hyperhomocysteinemia and possibly C. pneumoniae infection.

Unfortunately, vascular intervention, including angioplasty, stenting, atherectomy and grafting is often complicated by endothelial and smooth muscle cell proliferation resulting in restenosis or re-clogging of the artery. This may be due to endothelial cell injury caused by the treatment itself. Treatment of restenosis often involves a second angioplasty or bypass surgery. The drawbacks of such treatment are obvious including the risk of repeated restenosis.

In terms of the biological mechanism and characteristics leading to restenosis, accumulation of an extracellular matrix containing collagen and proteoglycans in association with smooth muscle cells characterizes both the atheroma and the arterial hyperplastic lesions that lead to restenosis after balloon injury or clinical angioplasty.

Various therapies have been attempted for treating or preventing restenosis. For example, the administration of multivitamins having antioxidant properties (30,000 IU of beta carotene, 500 mg of vitamin C and 700 IU of vitamin E) and/or probucol (500 mg) has been studied. The vitamins were administered twice daily for four weeks prior and six months after angioplasty, Tardif et al., N. Engl. J Med.: 337(6): 365-72, 1997. The antioxidant vitamins alone had no effect. Probucol did reduce the rate of restenosis after angioplasty by almost 50%. However, probucol was removed from the U.S. market for reducing HDL cholesterol levels and causing heart rhythm disturbances that potentially lead to dangerous arrhythmias.

Intracoronary irradiation during angioplasty and stent implantation to reduce the instances of restenosis have likewise been studied. Limitations of these methods include, for example, handling stents filled with radioactive liquid (Re 188-radioactive rhenium).

Clearly, there remains a great need for therapies useful for the prevention and treatment of atherosclerosis, restenosis and related disorders.

2.2 c-Jun N-Terminal Kinase

Three JNK enzymes have been identified. These represent alternatively spliced forms of three different genes: JNK1, JNK2, and JNK3 (Hibi M., Lin A., Smeal T., Minden A., Karin M. Genes Dev. 7:2135-2148, 1993; Mohit A. A., Martin M. H., and Miller C. A. Neuron 14:67-78, 1995; Gupta, S., Barrett, T., Whitmarsh, A. J., Cavanagh, J., Sluss, H. K., Derijard, B. and Davis, R. J. The EMBO J. 15:2760-2770, 1996). Activation of the JNK pathway has been documented in a number of disease settings, providing the rationale for targeting this pathway for drug discovery. In addition, molecular genetic approaches have validated the pathogenic role of the JNK pathway in several diseases.

The JNK pathway regulates TNF-α production in bacterial lipopolysaccharide-stimulated macrophages, and in mast cells stimulated through the FceRII receptor (Swantek J. L., Cobb M. H., Geppert T. D. Mol. Cell. Biol. 17:6274-6282, 1997; Ishizuka T., Tereda N., Gerwins P., Hamelmann E., Oshiba A., Fanger G. R., Johnson G. L., and Gelfland E. W. Proc. Nat. Acad. Sci. USA 94:6358-6363, 1997). Inhibition of JNK activation effectively modulates TNF-α secretion from these cells. Therefore, the JNK pathway regulates production of this key pro-inflammatory cytokine. Activated endothelial cells and smooth muscle cells both elaborate the B and T cell activator IL-6. IL-6 accounts for almost 4% of the newly synthesized proteins secreted by smooth muscle cells stimulated by IL-1. Human vascular wall cells also produce the monocyte chemoattractant and activator monocyte chemoattractant protein-1 (MCP-1)/JE (also known as macrophage chemoattractant and activating factor) and the monocyte differentiation and activating factor M-CSF (a macrophage colony stimulating factor). Accordingly, without being limited by theory, inhibition of JNK can limit the production of M-CSF and provide an effective method for treating or preventing atherosclerosis or restinosis.

3. SUMMARY OF THE INVENTION

In one embodiment, the present invention relates to a stent comprising an effective amount of a JNK Inhibitor, the stent (the “Stent of the Invention”) being useful for treating or preventing a cardiovascular or renal disease. In one embodiment, the Stent of the Invention comprises a coating comprising an effective amount of a JNK Inhibitor (the “coating”). In another embodiment, the stent comprises a material having an effective amount of a JNK Inhibitor incorporated therein (the “material”).

In another embodiment, the present invention encompasses a method for making a Stent of the Invention comprising the step of coating a stent with an effective amount of a JNK Inhibitor.

In another embodiment, the present invention encompasses a method for making a Stent of the Invention comprising the step of manufacturing a stent using a material having an effective amount of a JNK Inhibitor incorporated therein.

In another embodiment, the present invention encompasses methods for treating or preventing a cardiovascular or renal disease, comprising implanting a Stent of the Invention into a patient in need thereof.

In another embodiment, the present invention encompasses a kit comprising a Stent of the Invention and directions for its use.

The following Detailed Description and Examples illustrate non-limiting embodiments of the invention.

3.1 Definitions

As used herein, the term “patient” means an animal (e.g., cow, horse, sheep, pig, chicken, turkey, quail, cat, dog, mouse, rat, rabbit or guinea pig), preferably a mammal such as a non-primate or a primate (e.g., monkey or human), most preferably a human.

“Alkyl” means a saturated straight chain or branched non-cyclic hydrocarbon having from 1 to 10 carbon atoms. “Lower alkyl” means alkyl, as defined above, having from 1 to 4 carbon atoms. Representative saturated straight chain alkyls include -methyl, -ethyl, -n-propyl, -n-butyl, -n-pentyl, -n-hexyl, -n-heptyl, -n-octyl, -n-nonyl and -n-decyl; while saturated branched alkyls include -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like.

An “alkenyl group” or “alkylidene” mean a straight chain or branched non-cyclic hydrocarbon having from 2 to 10 carbon atoms and including at least one carbon-carbon double bond. Representative straight chain and branched (C₂-C₁₀)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl, -1-nonenyl, -2-nonenyl, -3-nonenyl, -1-decenyl, -2-decenyl, -3-decenyl and the like. An alkenyl group can be unsubstituted or substituted. A “cyclic alkylidene” is a ring having from 3 to 8 carbon atoms and including at least one carbon-carbon double bond, wherein the ring can have from 1 to 3 heteroatoms.

An “alkynyl group” means a straight chain or branched non-cyclic hydrocarbon having from 2 to 10 carbon atoms and including at lease one carbon-carbon triple bond. Representative straight chain and branched —(C₂-C₁₀)alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl, -1-heptynyl, -2-heptynyl, -6-heptynyl, -1-octynyl, -2-octynyl, -7-octynyl, -1-nonynyl, -2-nonynyl, -8-nonynyl, -1-decynyl, -2-decynyl, -9-decynyl, and the like. An alkynyl group can be unsubstituted or substituted.

The terms “Halogen” and “Halo” mean fluorine, chlorine, bromine or iodine.

“Haloalkyl” means an alkyl group, wherein alkyl is defined above, substituted with one or more halogen atoms.

“Keto” means a carbonyl group (i.e., C═O).

“Acyl” means an —C(O)alkyl group, wherein alkyl is defined above, including —C(O)CH₃, —C(O)CH₂CH₃, —C(O)(CH₂)₂CH₃, —C(O)(CH₂)₃CH₃, —C(O)(CH₂)₄CH₃, —C(O)(CH₂)₅CH₃, and the like.

“Acyloxy” means an —OC(O)alkyl group, wherein alkyl is defined above, including —OC(O)CH₃, —OC(O)CH₂CH₃, —OC(O)(CH₂)₂CH₃, —OC(O)(CH₂)₃CH₃, —OC(O)(CH₂)₄CH₃, —OC(O)(CH₂)₅CH₃, and the like.

“Ester” means and —C(O)Oalkyl group, wherein alkyl is defined above, including —C(O)OCH₃, —C(O)OCH₂CH₃, —C(O)O(CH₂)₂CH₃, —C(O)O(CH₂)₃CH₃, —C(O)O(CH₂)₄CH₃, —C(O)O(CH₂)₅CH₃, and the like.

“Alkoxy” means —O-(alkyl), wherein alkyl is defined above, including —OCH₃, —OCH₂CH₃, —O(CH₂)₂CH₃, —O(CH₂)₃CH₃, —O(CH₂)₄CH₃, —O(CH₂)₅CH₃, and the like. “Lower alkoxy” means —O-(lower alkyl), wherein lower alkyl is as described above.

“Alkoxyalkoxy” means —O-(alkyl)-O-(alkyl), wherein each alkyl is independently an alkyl group defined above, including —OCH₂OCH₃, —OCH₂CH₂OCH₃, —OCH₂CH₂OCH₂CH₃, and the like.

“Alkoxycarbonyl” means —C(═O)O-(alkyl), wherein alkyl is defined above, including —C(═O)O—CH₃, —C(═O)O—CH₂CH₃, —C(═O)O—(CH₂)₂CH₃, —C(═O)O—(CH₂)₃CH₃, —C(═O)O—(CH₂)₄CH₃, —C(═O)O—(CH₂)₅CH₃, and the like.

“Alkoxycarbonylalkyl” means -(alkyl)-C(═O)O-(alkyl), wherein each alkyl is independently defined above, including —CH₂—C(═O)O—CH₃, —CH₂—C(═O)O—CH₂CH₃, —CH₂—C(═O)O—(CH₂)₂CH₃, —CH₂—C(═O)O—(CH₂)₃CH₃, —CH₂—C(═O)O—(CH₂)₄CH₃, —CH₂—C(═O)O—(CH₂)₅CH₃, and the like.

“Alkoxyalkyl” means -(alkyl)-O-(alkyl), wherein each alkyl is independently an alkyl group defined above, including —CH₂OCH₃, —CH₂OCH₂CH₃, —(CH₂)₂OCH₂CH₃, —(CH₂)₂O(CH₂)₂CH₃, and the like.

“Aryl” means a carbocyclic aromatic group containing from 5 to 10 ring atoms. Representative examples include, but are not limited to, phenyl, tolyl, anthracenyl, fluorenyl, indenyl, azulenyl, pyridinyl and naphthyl, as well as benzo-fused carbocyclic moieties including 5,6,7,8-tetrahydronaphthyl. A carbocyclic aromatic group can be unsubstituted or substituted. In one embodiment, the carbocyclic aromatic group is a phenyl group.

“Aryloxy” means —O-aryl group, wherein aryl is as defined above. An aryloxy group can be unsubstituted or substituted. In one embodiment, the aryl ring of an aryloxy group is a phenyl group

“Arylalkyl” means -(alkyl)-(aryl), wherein alkyl and aryl are as defined above, including —(CH₂)phenyl, —(CH₂)₂phenyl, —(CH₂)₃phenyl, —CH(phenyl)₂, —CH(phenyl)₃, —(CH₂)tolyl, —(CH₂)anthracenyl, —(CH₂)fluorenyl, —(CH₂)indenyl, —(CH₂)azulenyl, —(CH₂)pyridinyl, —(CH₂)naphthyl, and the like.

“Arylalkyloxy” means —O-(alkyl)-(aryl), wherein alkyl and aryl are defined above, including —O—(CH₂)₂phenyl, —O—(CH₂)₃phenyl, —O—CH(phenyl)₂, —O—CH(phenyl)₃, —O—(CH₂)tolyl, —O—(CH₂)anthracenyl, —O—(CH₂)fluorenyl, —O—(CH₂)indenyl, —O—(CH₂)azulenyl, —O—(CH₂)pyridinyl, —O—(CH₂)naphthyl, and the like.

“Aryloxyalkyl” means -(alkyl)-O-(aryl), wherein alkyl and aryl are defined above, including —CH₂—O-(phenyl), —(CH₂)₂—O-phenyl, —(CH₂)₃—O-phenyl, —(CH₂)—O-tolyl, —(CH₂)—O-anthracenyl, —(CH₂)—O-fluorenyl, —(CH₂)—O-indenyl, —(CH₂)—O-azulenyl, —(CH₂)—O-pyridinyl, —(CH₂)—O-naphthyl, and the like.

“Cycloalkyl” means a monocyclic or polycyclic saturated ring having carbon and hydrogen atoms and having no carbon-carbon multiple bonds. Examples of cycloalkyl groups include, but are not limited to, (C₃-C₇)cycloalkyl groups, including cyclopropyl, cyclobutyl, cyclopentyl, cyclohexyl, and cycloheptyl, and saturated cyclic and bicyclic terpenes. A cycloalkyl group can be unsubstituted or substituted. In one embodiment, the cycloalkyl group is a monocyclic ring or bicyclic ring.

“Cycloalkyloxy” means —O-(cycloalkyl), wherein cycloalkyl is defined above, including —O-cyclopropyl, —O-cyclobutyl, —O-cyclopentyl, —O-cyclohexyl, —O-cycloheptyl and the like.

“Cycloalkylalkyloxy” means —O-(alkyl)-(cycloalkyl), wherein cycloalkyl and alkyl are defined above, including —O—CH₂-cyclopropyl, —O—(CH₂)₂-cyclopropyl, —O—(CH₂)₃-cyclopropyl, —O—(CH₂)₄-cyclopropyl, O—CH₂-cyclobutyl, O—CH₂-cyclopentyl , O—CH₂-cyclohexyl, O—CH₂-cycloheptyl, and the like.

“Aminoalkoxy” means —O-(alkyl)-NH₂, wherein alkyl is defined above, such as —O—CH₂—NH₂, —O—(CH₂)₂—NH₂, —O—(CH₂)₃—NH₂, —O—(CH₂)₄—NH₂, —O—(CH₂)₅—NH₂, and the like.

“Mono-alkylamino” means —NH(alkyl), wherein alkyl is defined above, such as —NHCH₃, —NHCH₂CH₃, —NH(CH₂)₂CH₃, —NH(CH₂)₃CH₃, —NH(CH₂)₄CH₃, —NH(CH₂)₅CH₃, and the like.

“Di-alkylamino” means —N(alkyl)(alkyl), wherein each alkyl is independently an alkyl group defined above, including —N(CH₃)₂, —N(CH₂CH₃)₂, —N((CH₂)₂CH₃)₂, —N(CH₃)(CH₂CH₃), and the like.

“Mono-alkylaminoalkoxy” means —O-(alkyl)-NH(alkyl), wherein each alkyl is independently an alkyl group defined above, including —O—(CH₂)—NHCH₃, —O—(CH₂)—NHCH₂CH₃, —O—(CH₂)—NH(CH₂)₂CH₃, —O—(CH₂)—NH(CH₂)₃CH₃, —O—(CH₂)—NH(CH₂)₄CH₃, —O—(CH₂)—NH(CH₂)₅CH₃, —O—(CH₂)₂—NHCH₃, and the like.

“Di-alkylaminoalkoxy” means —O-(alkyl)-N(alkyl)(alkyl), wherein each alkyl is independently an alkyl group defined above, including —O—(CH₂)—N(CH₃)₂, —O—(CH₂)—N(CH₂CH₃)₂, —O—(CH₂)—N((CH₂)₂CH₃)₂, —O—(CH₂)—N(CH₃)(CH₂CH₃), and the like.

“Arylamino” means —NH(aryl), wherein aryl is defined above, including —NH(phenyl), —NH(tolyl), —NH(anthracenyl), —NH(fluorenyl), —NH(indenyl), —NH(azulenyl), —NH(pyridinyl), —NH(naphthyl), and the like.

“Arylalkylamino” means —NH-(alkyl)-(aryl), wherein alkyl and aryl are defined above, including —NH—CH₂-(phenyl), —NH—CH₂-(tolyl), —NH—CH₂-(anthracenyl), —NH—CH₂-(fluorenyl), —NH—CH₂-(indenyl), —NH—CH₂-(azulenyl), —NH—CH₂-(pyridinyl), —NH—CH₂-(naphthyl), —NH—(CH₂)₂-(phenyl) and the like.

“Alkylamino” means mono-alkylamino or di-alkylamino as defined above, such as -N(alkyl)(alkyl), wherein each alkyl is independently an alkyl group defined above, including —N(CH₃)₂, —N(CH₂CH₃)₂, —N((CH₂)₂CH₃)₂, —N(CH₃)(CH₂CH₃) and —N(alkyl)(alkyl), wherein each alkyl is independently an alkyl group defined above, including —N(CH₃)₂, —N(CH₂CH₃)₂, —N((CH₂)₂CH₃)₂, —N(CH₃)(CH₂CH₃) and the like.

“Cycloalkylamino” means —NH-(cycloalkyl), wherein cycloalkyl is as defined above, including —NH-cyclopropyl, —NH-cyclobutyl, —NH-cyclopentyl, —NH-cyclohexyl, —NH-cycloheptyl, and the like.

“Carboxyl” and “carboxy” mean —COOH.

“Cycloalkylalkylamino” means —NH-(alkyl)-(cycloalkyl), wherein alkyl and cycloalkyl are defined above, including —NH—CH₂-cyclopropyl, —NH—CH₂-cyclobutyl, —NH—CH₂-cyclopentyl, —NH—CH₂-cyclohexyl, —NH—CH₂-cycloheptyl, —NH—(CH₂)₂-cyclopropyl and the like.

“Aminoalkyl” means -(alkyl)-NH₂, wherein alkyl is defined above, including CH₂—NH₂, —(CH₂)₂—NH₂, —(CH₂)₃—NH₂, —(CH₂)₄—NH₂, —(CH₂)₅—NH₂ and the like.

“Mono-alkylaminoalkyl” means -(alkyl)-NH(alkyl),wherein each alkyl is independently an alkyl group defined above, including —CH₂—NH—CH₃, —CH₂—NHCH₂CH₃, —CH₂—NH(CH₂)₂CH₃, —CH₂—NH(CH₂)₃CH₃, —CH₂—NH(CH₂)₄CH₃, —CH₂—NH(CH₂)₅CH₃, —(CH₂)₂—NH—CH₃, and the like. “Di-alkylaminoalkyl” means -(alkyl)-N(alkyl)(alkyl),wherein each alkyl is independently an alkyl group defined above, including —CH₂—N(CH₃)₂, —CH₂—N(CH₂CH₃)₂, —CH₂—N((CH₂)₂CH₃)₂, —CH₂—N(CH₃)(CH₂CH₃), —(CH₂)₂—N(CH₃)₂, and the like.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are triazolyl, tetrazolyl, oxadiazolyl, pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, quinazolinyl, pyrimidyl, oxetanyl, azepinyl, piperazinyl, morpholinyl, dioxanyl, thietanyl and oxazolyl.

“Heteroarylalkyl” means -(alkyl)-(heteroaryl), wherein alkyl and heteroaryl are defined above, including —CH₂-triazolyl, —CH₂-tetrazolyl, —CH₂-oxadiazolyl, —CH₂-pyridyl, —CH₂-furyl, —CH₂-benzofuranyl, —CH₂-thiophenyl, —CH₂-benzothiophenyl, —CH₂-quinolinyl, —CH₂-pyrrolyl, —CH₂-indolyl, —CH₂-oxazolyl, —CH₂-benzoxazolyl, —CH₂-imidazolyl, —CH₂-benzimidazolyl, —CH₂-thiazolyl, —CH₂-benzothiazolyl, —CH₂-isoxazolyl, —CH₂-pyrazolyl, —CH₂-isothiazolyl, —CH₂-pyridazinyl, —CH₂-pyrimidinyl, —CH₂-pyrazinyl, —CH₂-triazinyl, —CH₂-cinnolinyl, —CH₂-phthalazinyl, —CH₂-quinazolinyl, —CH₂-pyrimidyl, —CH₂-oxetanyl, —CH₂-azepinyl, —CH₂-piperazinyl, —CH₂-morpholinyl, —CH₂-dioxanyl, —CH₂-thietanyl, —CH₂-oxazolyl, —(CH₂)₂-triazolyl, and the like.

“Heterocycle” means a 5- to 7-membered monocyclic, or 7- to 10-membered bicyclic, heterocyclic ring which is either saturated, unsaturated, and which contains from 1 to 4 heteroatoms independently selected from nitrogen, oxygen and sulfur, and wherein the nitrogen and sulfur heteroatoms can be optionally oxidized, and the nitrogen heteroatom can be optionally quaternized, including bicyclic rings in which any of the above heterocycles are fused to a benzene ring. The heterocycle can be attached via any heteroatom or carbon atom. Heterocycles include heteroaryls as defined above. Representative heterocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heterocycle fused to phenyl” means a heterocycle, wherein heterocycle is defined as above, that is attached to a phenyl ring at two adjacent carbon atoms of the phenyl ring.

“Heterocycloalkyl” means -(alkyl)-(heterocycle), wherein alkyl and heterocycle are defined above, including —CH₂-morpholinyl, —CH₂-pyrrolidinonyl, —CH₂-pyrrolidinyl, —CH₂-piperidinyl, —CH₂-hydantoinyl, —CH₂-valerolactamyl, —CH₂-oxiranyl, —CH₂-oxetanyl, —CH₂-tetrahydrofuranyl, —CH₂-tetrahydropyranyl, —CH₂-tetrahydropyridinyl, —CH₂-tetrahydroprimidinyl, —CH₂-tetrahydrothiophenyl, —CH₂-tetrahydrothiopyranyl, —CH₂-tetrahydropyrimidinyl, —CH₂-tetrahydrothiophenyl, —CH₂-tetrahydrothiopyranyl, and the like.

The term “substituted” as used herein means any of the above groups (i.e., aryl, arylalkyl, heterocycle and heterocycloalkyl) wherein at least one hydrogen atom of the moiety being substituted is replaced with a substituent. In one embodiment, each carbon atom of the group being substituted is substituted with no more that two substituents. In another embodiment, each carbon atom of the group being substituted is substituted with no more than one substituent. In the case of a keto substituent, two hydrogen atoms are replaced with an oxygen which is attached to the carbon via a double bond. Substituents include halogen, hydroxyl, alkyl, haloalkyl, mono- or di-substituted aminoalkyl, alkyloxyalkyl, aryl, arylalkyl, heterocycle, heterocycloalkyl, —NRₐR_{b}, —NRₐC(═O)R_{b}, —NRₐC(═O)NRₐR_{b}, —NRₐC(═O)OR_{b}—NRₐSO₂R_{b}, —ORₐ, —C(═O)Rₐ C(═O)ORₐ—C(═O)NRₐR_{b}, —OC(═O)Rₐ, —OC(═O)ORₐ, —OC(═O)NRₐR_{b}, —NRₐSO₂R_{b}, or a radical of the formula —Y—Z—Rₐ where Y is alkanediyl, or a direct bond, Z is —O—, —S—, —N(R_{b})—, —C(═O)—, —C(═O)O—, —OC(═O)—, —N(R_{b})C(═O)—, —C(═O)N(R_{b})— or a direct bond, wherein Rₐ and R_{b} are the same or different and independently hydrogen, amino, alkyl, haloalkyl, aryl, arylalkyl, heterocycle, or heterocylealkyl, or wherein Rₐ and R_{b} taken together with the nitrogen atom to which they are attached form a heterocycle.

“Haloalkyl” means alkyl, wherein alkyl is defined as above, having one or more hydrogen atoms replaced with halogen, wherein halogen is as defined above, including —CF₃, —CHF₂, —CH₂F, —CBr₃, —CHBr₂, —CH₂Br, —CCl₃, —CHCl₂, —CH₂Cl, —CI₃, —CHI₂, —CH₂I, —CH₂—CF₃, —CH₂—CHF₂, —CH₂—CH₂F, —CH₂—CBr₃, —CH₂—CHBr₂, —CH₂—CH₂Br, —CH₂—CCl₃, —CH₂—CHCl₂, —CH₂—CH₂Cl, —CH₂—CI₃, —CH₂—CHI₂, —CH₂—CH₂I, and the like.

“Hydroxyalkyl” means alkyl, wherein alkyl is as defined above, having one or more hydrogen atoms replaced with hydroxy, including —CH₂OH, —CH₂CH₂OH, —(CH₂)₂CH₂OH, —(CH₂)₃CH₂OH, —(CH₂)₄CH₂OH, —(CH₂)₅CH₂OH, —CH(OH)—CH₃, —CH₂CH(OH)CH₃, and the like.

“Hydroxy” means —OH.

“Sulfonyl” means —SO₃H.

“Sulfonylalkyl” means —SO₂-(alkyl), wherein alkyl is defined above, including —SO₂—CH₃, —SO₂—CH₂CH₃, —SO₂—(CH₂)₂CH₃, —SO₂—(CH₂)₃CH₃, —SO₂—(CH₂)₄CH₃, —SO₂—(CH₂)₅CH₃, and the like.

“Sulfinylalkyl” means —SO-(alkyl), wherein alkyl is defined above, including —SO—CH₃, —SO—CH₂CH₃, —SO—(CH₂)₂CH₃, —SO—(CH₂)₃CH₃, —SO—(CH₂)₄CH₃, —SO—(CH₂)₅CH₃, and the like.

“Sulfonamidoalkyl” means —NHSO₂-(alkyl), wherein aklyl is defined above, including —NHSO₂—CH₃, —NHSO₂—CH₂CH₃, —NHS₂—(CH₂)₂CH₃, —NHSO₂—(CH₂)₃CH₃, —NHSO₂—(CH₂)₄CH₃, —NHSO₂—(CH₂)₅CH₃, and the like.

“Thioalkyl” means —S-(alkyl), wherein alkyl is defined above, including —S—CH₃, —S—CH₂CH₃, —S—(CH₂)₂CH₃, —S—(CH₂)₃CH₃, —S—(CH₂)₄CH₃, —S—(CH₂)₅CH₃, and the like.

As used herein, the term “JNK Inhibitor” encompasses, but is not limited to, compounds disclosed herein. Without being limited by theory, specific JNK Inhibitors are capable of inhibiting the activity of JNK in vitro or in vivo. The JNK Inhibitor can be in the form of a pharmaceutically acceptable salt, free base, solvate, hydrate, stereoisomer, clathrate or prodrug thereof. Such inhibitory activity can be determined by an assay or animal model well-known in the art including those set forth in Section 5.2. In one embodiment, the JNK Inhibitor is a compound of structure (I)-(III).

As used herein, a “Stent of the Invention” means any device useful for opening up an artery, vein or capillary thereby improving blood flow; keeping an artery, vein or capillary open; sealing any tears or openings in an artery, vein or capillary; preventing an artery, vein or capillary wall from collapsing or closing off again; or preventing small pieces of plaque from breaking off. In one embodiment, the stent is a stent graft.

As used herein, a “stent graft” means any stent that is covered with a synthetic or natural material to form a graft prosthesis. The term also encompasses grafted stents, wherein the stent is covered in its entirety with a natural or synthetic graft material (e.g., Vanguard-graft stent, Palmaz-Impragraft stent or Corvita stent). In one embodiment, the stent graft is a prosthetic.

An “effective amount” when used in connection with a JNK Inhibitor is an amount of the JNK Inhibitor that is useful for treating or preventing a cardiovascular or renal disease.

An “effective amount” when used in connection with another active agent is an amount of the other active agent that is useful for providing the agent's therapeutic or prophylactic effect while the JNK Inhibitor is exerting its therapeutic or prophylactic effect.

When coated, the coating can be present on any portion of a surface of the stent. In one embodiment, the surface is the inner surface. In another embodiment, the surface is the outer surface. In one embodiment, the layer covers at least about 10% of the surface. In another embodiment, the layer covers at least about 20% of the surface. In another embodiment, the layer covers at least about 30% of the surface. In another embodiment, the layer covers at least about 40% of the surface. In another embodiment, the layer covers at least about 50% of the surface. In another embodiment, the layer covers at least about 60% of the surface. In another embodiment, the layer covers at least about 70% of the surface. In another embodiment, the layer covers at least about 80% of the surface. In another embodiment, the layer covers at least about 90% of the surface. In another embodiment, the layer covers about 100% of the surface.

As used herein, the term “preventing” includes inhibiting a cardiovascular or renal disease, in particular, atherosclerosis, stenosis or restinosis or a symptom of atherosclerosis, stenosis or restinosis.

As used herein, the term “treating” includes eradicating a cardiovascular or renal disease, in particular, atherosclerosis, stenosis or restinosis or a symptom of atherosclerosis, stenosis or restinosis. In one embodiment, “treating” refers to minimizing the spread or minimizing the worsening of a cardiovascular or renal disease, in particular, atherosclerosis, stenosis or restinosis or a symptom of atherosclerosis, stenosis or restinosis.

“JNK” means a protein or an isoform thereof expressed by a JNK 1, JNK 2, or JNK 3 gene (Gupta, S., Barrett, T., Whitmarsh, A. J., Cavanagh, J., Sluss, H. K., Derijard, B. and Davis, R. J. The EMBO J 15:2760-2770 (1996)).

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the JNK Inhibitor include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art, see for example, Remington's Pharmaceutical Sciences, 18^{th} eds., Mack Publishing, Easton Pa. (1990) or Remington: The Science and Practice of Pharmacy, 19^{th} eds., Mack Publishing, Easton Pa. (1995).

As used herein, the term “polymorph(s)” and related terms herein refer to solid forms of the JNK Inhibitor having different physical properties as a result of the order of the molecules in the crystal lattice. The differences in physical properties exhibited by solid forms affect pharmaceutical parameters such as storage stability, compressibility and density (important in formulation and product manufacturing), and dissolution rates (an important factor in determining bioavailability). Differences in stability can result from changes in chemical reactivity (e.g., differential oxidation, such that a dosage form discolors more rapidly when comprised of one solid form than when comprised of another solid form) or mechanical changes (e.g., tablets crumble on storage as a kinetically favored polymorph converts to thermodynamically more stable solid form) or both (e.g., tablets of one solid form are more susceptible to breakdown at high humidity). As a result of solubility/dissolution differences, in the extreme case, some solid form transitions may result in lack of potency or, at the other extreme, toxicity. In addition, the physical properties of the crystal may be important in processing, for example, one solid form might be more likely to form solvates or might be difficult to filter and wash free of impurities (i.e., particle shape and size distribution might be different between one solid form relative to the other).

As used herein and unless otherwise indicated, the term “clathrate” means a JNK Inhibitor, or a salt thereof, in the form of a crystal lattice that contains spaces (e.g., channels) that have a guest molecule (e.g., a solvent or water) trapped within.

As used herein and unless otherwise indicated, the term “hydrate” means a JNK Inhibitor, or a salt thereof, that further includes a stoichiometric or non-stoichiometric amount of water bound by non-covalent intermolecular forces.

As used herein and unless otherwise indicated, the term “prodrug” means a JNK Inhibitor derivative that can hydrolyze, oxidize, or otherwise react under biological conditions (in vitro or in vivo) to provide an active compound, particularly a JNK Inhibitor. Examples of prodrugs include, but are not limited to, derivatives and metabolites of a JNK Inhibitor that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Preferably, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a JNK Inhibitor that is substantially free of other stereoisomers of that compound. For example, a stereomerically pure compound having one chiral center will be substantially free of the opposite enantiomer of the compound. A stereomerically pure a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

### 4. DETAILED DESCRIPTION OF THE INVENTION

In one embodiment, the present invention encompasses a Stent of the Invention useful for treating or preventing a cardiovascular or renal disease.

In another embodiment, the present invention encompasses methods for treating or preventing a cardiovascular or renal disease, including atherosclerosis, and in particular, the treatment or prevention of restenosis after vascular intervention such as angioplasty, comprising implanting into a patient in need thereof a Stent of the Invention.

In another embodiment, the present invention encompasses a method for making a Stent of the Invention, comprising the step of coating a stent with an effective amount of a JNK Inhibitor. The coating step can include dipping, spraying, casting, layering, adding to or filling a stent with an effective amount of one or more JNK Inhibitors.

In another embodiment, the present invention encompasses a method for making a Stent of the Invention, comprising the step of manufacturing a stent using a material having an effective amount of a JNK Inhibitor incorporated therein.

In another embodiment, the Stent of the Invention further comprise an effective amount of another active agent useful for treating or preventing a cardiovascular or renal disease. Such active agents include, but are not limited to: an anticoagulant agent, an antimetabolite agent, an anti-inflammatory agent, an antiplatelet agent, an antithrombin agent, an antimitotic agent, a cytostatic agent and an antiproliferative agent (see Section 4.5 for further examples of other active agents).

In another embodiment, the Stent of the Invention further comprise nitric oxide.

In another embodiment, the Stent of the Invention further comprise an antibiotic agent or an antiviral agent, or mixtures thereof, which can prevent graft rejection.

In one embodiment, the coating comprises a plurality of layers.

In one embodiment, the coating is a controlled-release coating.

In one embodiment, the Stent of the Invention is comprised of material which allows for controlled-release of the JNK Inhibitor incorporated therein.

In another embodiment, the present invention encompasses a kit comprising a Stent of the Invention and directions for its use.

4.1 Illustrative JNK Inhibitors

As mentioned above, the present invention is directed to methods useful for treating or preventing a cardiovascular or renal disease, comprising implanting into a patient in need thereof a Stent of the Invention (i.e., a stent comprising an effective amount of a JNK Inhibitor). Illustrative JNK Inhibitors are set forth below.

In one embodiment, the JNK Inhibitor has the following structure (I): 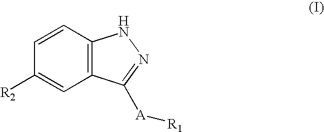
wherein:
A is a direct bond, —(CH₂)ₐ—, —(CH₂)_{b}CH═CH(CH₂)_{c}—, or —(CH₂)_{b}C≡C(CH₂)_{c}—;
R₁ is aryl, heteroaryl or heterocycle fused to phenyl, each being optionally substituted with one to four substituents independently selected from R₃;
R₂ is —R₃, —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)_{c}C(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R₆;
a is 1, 2, 3, 4, 5 or 6;
b and c are the same or different and at each occurrence independently selected from 0, 1, 2, 3 or 4;
d is at each occurrence 0, 1 or 2;
R₃ is at each occurrence independently halogen, hydroxy, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₈OR₉, —SO₂NR₈R₉, —NR₈SO₂R₉, —CN, —NO₂, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₄ is alkyl, aryl, arylalkyl, heterocycle or heterocycloalkyl, each being optionally substituted with one to four substituents independently selected from R₃, or R₄ is halogen or hydroxy;
R₅, R₆ and R₇ are the same or different and at each occurrence independently hydrogen, alkyl, aryl, arylalkyl, heterocycle or heterocycloalkyl, wherein each of R₅, R₆ and R₇ are optionally substituted with one to four substituents independently selected from R₃; and
R₈ and R₉ are the same or different and at each occurrence independently hydrogen, alkyl, aryl, arylalkyl, heterocycle, or heterocycloalkyl, or R₈ and R₉ taken together with the atom or atoms to which they are bonded form a heterocycle, wherein each of R₈, R₉, and R₈ and R₉ taken together to form a heterocycle are optionally substituted with one to four substituents independently selected from R₃.

In one embodiment, -A-R₁ is phenyl, optionally substituted with one to four substituents independently selected from halogen, alkoxy, —NR₈C(═O)R₉, —C(═O)NR₈R₉, and —O(CH₂)_{b}NR₈R₉, wherein b is 2 or 3 and wherein R₈ and R₉ are defined above.

In another embodiment, R₂ is —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)_{c}C(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R₆, and b is an integer ranging from 0-4.

In another embodiment, R₂ is —(CH₂)_{b}C(═O)NR₅R₆, —(CH₂)_{b}NR₅C(═O)R₆, 3-triazolyl or 5-tetrazolyl, wherein b is 0 and wherein R₈ and R₉ are defined above.

In another embodiment, R₂ is 3-triazolyl or 5-tetrazolyl.

In another embodiment:
(a) -A-R₁ is phenyl, optionally substituted with one to four substituents independently selected from halogen, alkoxy, —NR₈C(═O)R₉, —C(═O)NR₈R₉,
and —O(CH₂)_{b}NR₈R₉, wherein b is 2 or 3; and
(b) R₂ is —(CH₂)_{b}C(═O)NR₅R₆, —(CH₂)_{b}NR₅C(═O)R₆, 3-triazolyl or 5-tetrazolyl, wherein b is 0 and wherein R₈ and R₉ are defined above.

In another embodiment:
(a) -A-R₁ is phenyl, optionally substituted with one to four substituents independently selected from halogen, alkoxy, —NR₈C(═O)R₉, —C(═O)NR₈R₉, and —O(CH₂)_{b}NR₈R₉, wherein b is 2 or 3; and
(b) R₂ is 3-triazolyl or 5-tetrazolyl.

In another embodiment, R₂ is R₄, and R₄ is 3-triazolyl, optionally substituted at its 5-position with:
(a) a C₁-C₄ straight or branched chain alkyl group optionally substituted with a hydroxyl, methylamino, dimethylamino or 1-pyrrolidinyl group; or
(b) a 2-pyrrolidinyl group.

In another embodiment, R₂ is R_{4,} and R₄ is 3-triazolyl, optionally substituted at its 5-position with: methyl, n-propyl, isopropyl, 1-hydroxyethyl, 3-hydroxypropyl, methylaminomethyl, dimethylaminomethyl, 1-(dimethylamino)ethyl, 1-pyrrolidinylmethyl or 2-pyrrolidinyl.

In another embodiment, the compounds of structure (I) have structure (IA) when A is a direct bond, or have structure (IB) when A is —(CH₂)ₐ—:

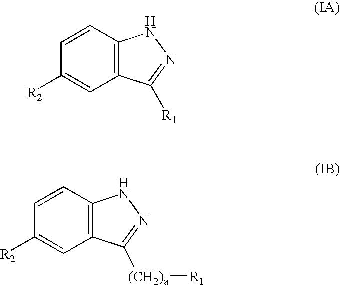

In other embodiments, the compounds of structure (I) have structure (IC) when A is a —CH₂)_{b}CH═CH(CH₂)_{c}—, and have structure (ID) when A is —(CH₂)_{b}C≡C(CH₂)_{c}—:

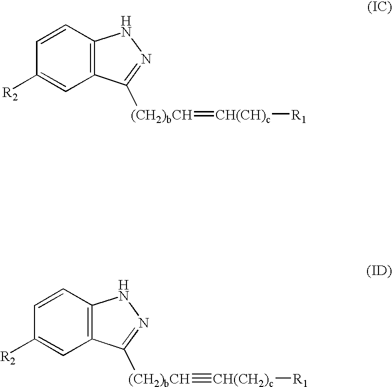

In further embodiments of this invention, R₁ of structure (I) is aryl or substituted aryl, such as phenyl or substituted phenyl as represented by the following structure (IE):

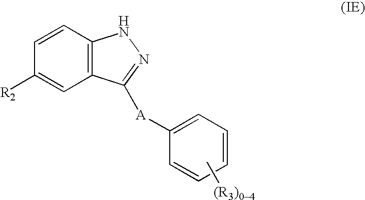

In another embodiment, R₂ of structure (I) is —(CH₂)_{b}NR₄(C═O)R₅. In one aspect of this embodiment, b=0 and the compounds have the following structure (IF):

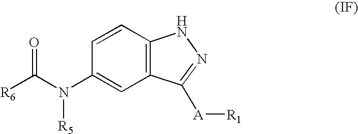

Representative R₂ groups of the compounds of structure (I) include alkyl (such as methyl and ethyl), halo (such as chloro and fluoro), haloalkyl (such as trifluoromethyl), hydroxy, alkoxy (such as methoxy and ethoxy), amino, arylalkyloxy (such as benzyloxy), mono- or di-alkylamine (such as —NHCH₃, —N(CH₃)₂ and —NHCH₂CH₃), —NHC(═O)R₄ wherein R₆ is a substituted or unsubstituted phenyl or heteroaryl (such as phenyl or heteroaryl substituted with hydroxy, carboxy, amino, ester, alkoxy, alkyl, aryl, haloalkyl, halo, —CONH₂ and —CONH alkyl), —NH(heteroarylalkyl) (such as —NHCH₂(3-pyridyl), —NHCH₂(4-pyridyl), heteroaryl (such as pyrazolo, triazolo and tetrazolo), —C(═O)NHR₆ wherein R₆ is hydrogen, alkyl, or as defined above (such as —C(═O)NH₂, —C(═O)NHCH₃, —C(═O)NH(H-carboxyphenyl), —C(═O)N(CH₃)₂), arylalkenyl (such as phenylvinyl, 3-nitrophenylvinyl, 4-carboxyphenylvinyl), heteroarylalkenyl (such as 2-pyridylvinyl, 4-pyridylvinyl).

Representative R₃ groups of the compounds of structure (I) include halogen (such as chloro and fluoro), alkyl (such as methyl, ethyl and isopropyl), haloalkyl (such as trifluoromethyl), hydroxy, alkoxy (such as methoxy, ethoxy, n-propyloxy and isobutyloxy), amino, mono- or di-alkylamino (such as dimethylamine), aryl (such as phenyl), carboxy, nitro, cyano, sulfinylalkyl (such as methylsulfinyl), sulfonylalkyl (such as methylsulfonyl), sulfonamidoalkyl (such as —NHSO₂CH₃), —NR₈C(═O)(CH₂)_{b}OR₉ (such as NHC(═O)CH₂OCH₃), NHC(═O)R₉ (such as —NHC(═O)CH₃, —NHC(═O)CH₂C₆H₅, —NHC(═O)(2-furanyl)), and —O(CH₂)_{b}NR₈R₉ (such as —O(CH₂)₂N(CH₃)₂).

The compounds of structure (I) can be made using organic synthesis techniques known to those skilled in the art, as well as by the methods described in International Publication No. WO 02/10137 (particularly in Examples 1-430, at page 35, line 1 to page 396, line 12), published Feb. 7, 2002, which is incorporated herein by reference in its entirety. Further, specific examples of these compounds are found in this publication.

Illustrative examples of JNK Inhibitors of structure (I) are: 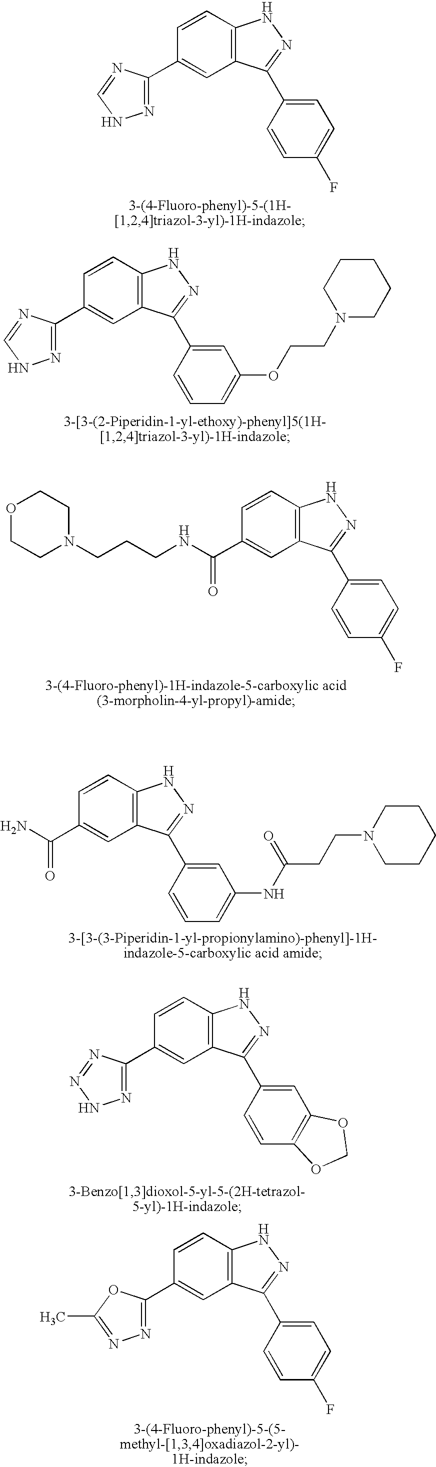 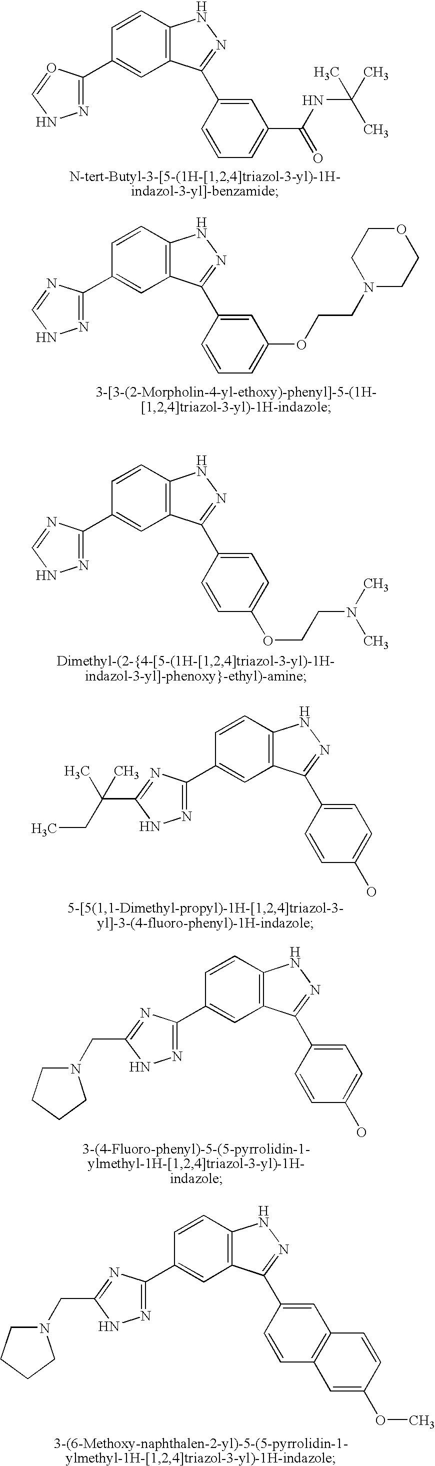 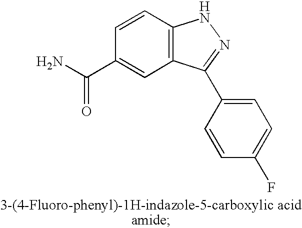
and pharmaceutically acceptable salts thereof.

In another embodiment, the JNK Inhibitor has the following structure (II): 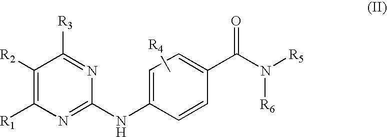
wherein:
R₁ is aryl or heteroaryl optionally substituted with one to four substituents independently selected from R₇;
R₂ is hydrogen;
R₃ is hydrogen or lower alkyl;
R₄ represents one to four optional substituents, wherein each substituent is the same or different and independently selected from halogen, hydroxy, lower alkyl and lower alkoxy;
R₅ and R₆ are the same or different and independently —R₈, —(CH₂)ₐC(═O)R₉, —(CH₂)ₐC(═O)OR₉, —(CH₂)ₐC(═O)NR₉R₁₀, —(CH₂)ₐC(═O)NR₉(CH₂)_{b}C(═O)R₁₀, —(CH₂)ₐNR₉C(═O)R₁₀, (CH₂)ₐNR₁₁C(═O)NR₉R₁₀, —(CH₂)ₐNR₉R₁₀, —(CH₂)ₐOR₉, —(CH₂)ₐSO_{c}R₉ or —(CH₂)ₐSO₂NR₉R₁₀;
or R₅ and R₆ taken together with the nitrogen atom to which they are attached to form a heterocycle or substituted heterocycle;
R₇ is at each occurrence independently halogen, hydroxy, cyano, nitro, carboxy, alkyl, alkoxy, haloalkyl, acyloxy, thioalkyl, sulfinylalkyl, sulfonylalkyl, hydroxyalkyl, aryl, arylalkyl, heterocycle, substituted heterocycle, heterocycloalkyl, _C(═O)OR₈, —OC(═O)R₈, —C(═O)NR₈R₉, —C(═O)NR₈OR₉, —SO_{c}R₈, —SO_{c}NR₈R₉, —NR₈SO_{c}R₉, —NR₈R₉, —NR₈C(═O)R₉, —NR₈C(═O)(CH₂)_{b}OR₉, —NR₈C(═O)(CH₂)_{b}R₉, _O(CH₂)_{b}NR₈R₉, or heterocycle fused to phenyl;
R₈, R₉, R₁₀ and R₁₁ are the same or different and at each occurrence independently hydrogen, alkyl, aryl, arylalkyl, heterocycle, heterocycloalkyl;
or R₈ and R₉ taken together with the atom or atoms to which they are attached to form a heterocycle;
a and b are the same or different and at each occurrence independently selected from 0, 1, 2, 3 or 4; and
c is at each occurrence 0, 1 or 2.

In one embodiment, R₁ is a substituted or unsubstituted aryl or heteroaryl. When R₁ is substituted, it is substituted with one or more substituents defined below. In one embodiment, when substituted, R₁ is substituted with a halogen, —SO₂R₈ or —SO₂R₈R₉.

In another embodiment, R₁ is substituted or unsubstituted aryl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl or quinazolinyl.

In another embodiment R₁ is substituted or unsubstituted aryl or heteroaryl. When R₁ is substituted, it is substituted with one or more substituents defined below. In one embodiment, when substituted, R₁ is substituted with a halogen, —SO₂R₈ or —SO₂R₈R₉.

In another embodiment, R₁ is substituted or unsubstituted aryl, preferably phenyl. When R₁ is a substituted aryl, the substituents are defined below. In one embodiment, when substituted, R₁ is substituted with a halogen, —SO₂R₈ or —SO₂R₈R₉.

In another embodiment, R₅ and R₆, taken together with the nitrogen atom to which they are attached form a substituted or unsubstituted nitrogen-containing non-aromatic heterocycle, in one embodiment, piperazinyl, piperidinyl or morpholinyl.

When R₅ and R₆, taken together with the nitrogen atom to which they are attached form substituted piperazinyl, piperadinyl or morpholinyl, the piperazinyl, piperadinyl or morpholinyl is substituted with one or more substituents defined below. In one embodiment, when substituted, the substituent is alkyl, amino, alkylamino, alkoxyalkyl, acyl, pyrrolidinyl or piperidinyl.

In one embodiment, R₃ is hydrogen and R₄ is not present, and the JNK Inhibitor has the following structure (IIA): 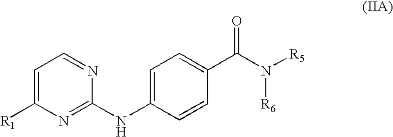
and pharmaceutically acceptable salts thereof.

In a more specific embodiment, R₁ is phenyl optionally substituted with R₇, and having the following structure (IIB): 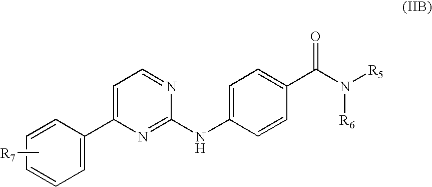
and pharmaceutically acceptable salts thereof.

In still a further embodiment, R₇ is at the para position of the phenyl group relative to the pyrimidine, as represented by the following structure (IIC): 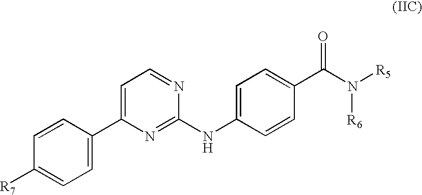
and pharmaceutically acceptable salts thereof.

The JNK Inhibitors of structure (II) can be made using organic synthesis techniques known to those skilled in the art, as well as by the methods described in International Publication No. WO 02/46170 (particularly Examples 1-27 at page 23, line 5 to page 183, line 25), published Jun. 13, 2002, which is hereby incorporated by reference in itsr entirety. Further, specific examples of these compounds are found in the publication.

Illustrative examples of JNK Inhibitors of structure (II) are: 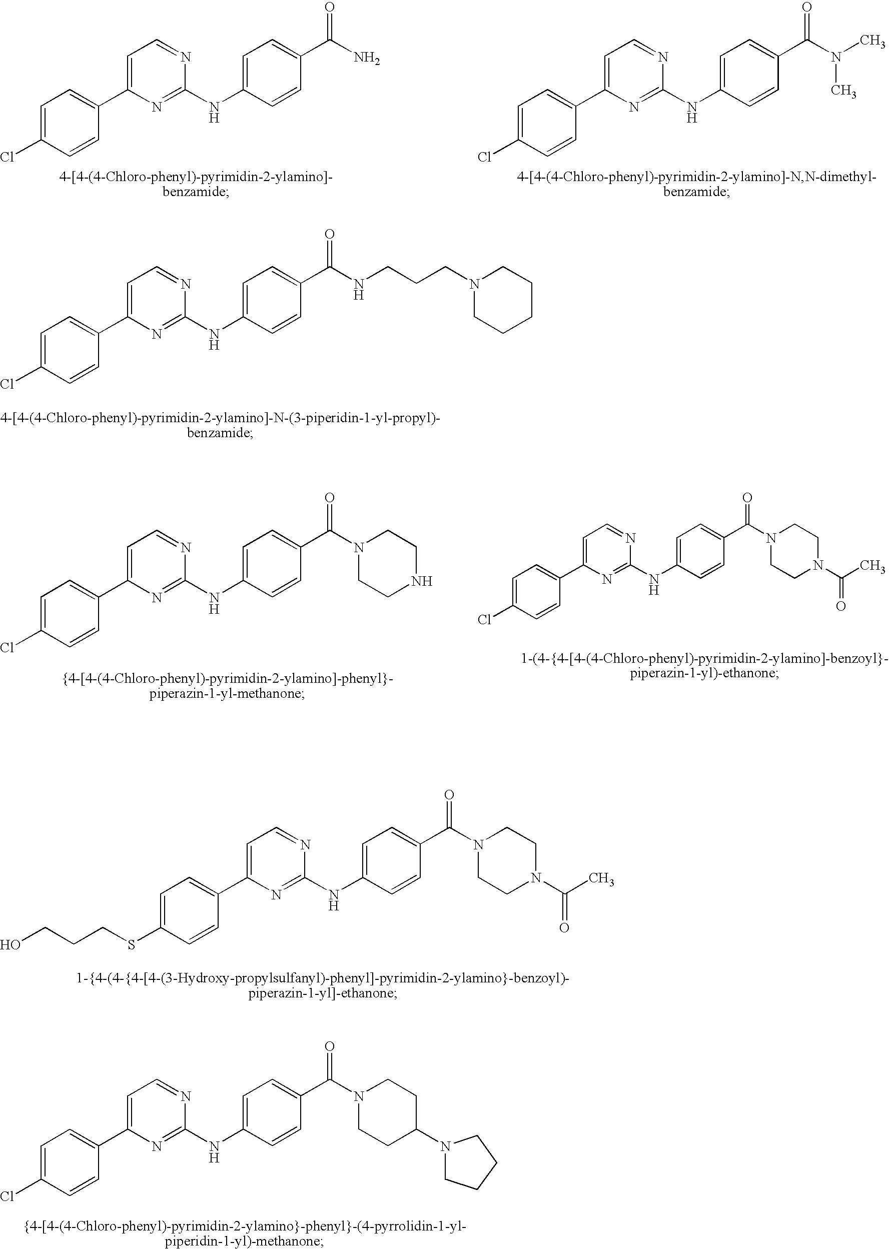
and pharmaceutically acceptable salts thereof.

In another embodiment, the JNK Inhibitor has the following structure (III): 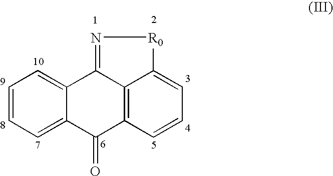
wherein R₀ is —O—, —S—, —S(O)—, —S(O)₂—, NH or —CH₂—;
the compound of structure (III) being: (i) unsubstituted, (ii) monosubstituted and having a first substituent, or (iii) disubstituted and having a first substituent and a second substituent;
the first or second substituent, when present, is at the 3, 4, 5, 7, 8, 9, or 10 position, wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f): 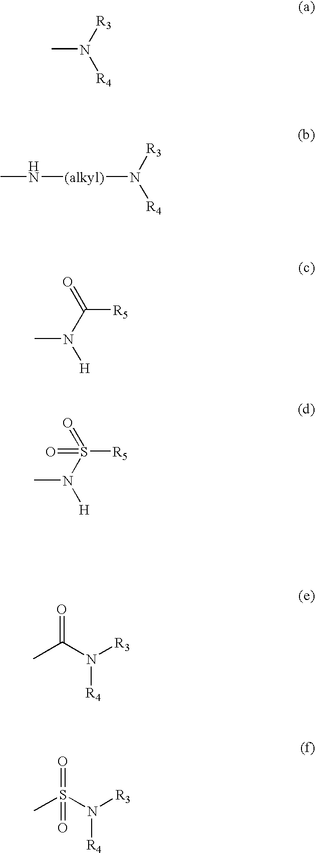
wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and
R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

In another embodiment, the JNK Inhibitor has the following structure (IIIA): 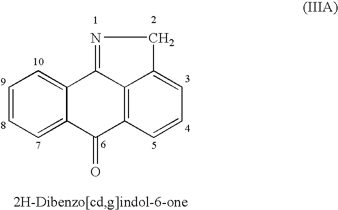
being: (i) unsubstituted, (ii) monosubstituted and having a first substituent, or (iii) disubstituted and having a first substituent and a second substituent;
the first or second substituent, when present, is at the 3, 4, 5, 7, 8, 9, or 10 position;
wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono- alkylaminoalkoxy, di-alkylaminoalkoxy, or a group represented by structure (a), (b), (c), (d), (e), or (f): 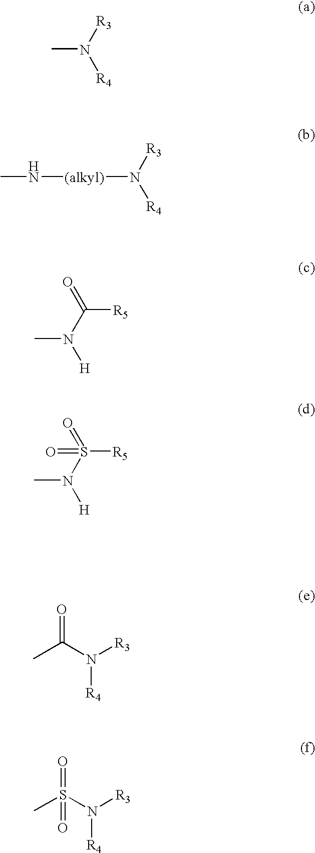
wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and
R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

A subclass of the compounds of structure (IIIA) is that wherein the first or second substituent is present at the 5, 7, or 9 position. In one embodiment, the first or second substituent is present at the 5 or 7 position.

A second subclass of compounds of structure (IIIA) is that wherein the first or second substituent is present at the 5, 7, or 9 position;
the first or second substituent is independently alkoxy, aryloxy, aminoalkyl, mono-alkylaminoalkyl, di-alkylaminoalkyl, or a group represented by the structure (a), (c), (d), (e), or (f);
R₃ and R₄ are independently hydrogen, alkyl, cycloalkyl, aryl, arylalkyl, or cycloalkylalkyl; and
R₅ is hydrogen, alkyl, cycloalkyl, aryl, arylalkyl, or cycloalkylalkyl.

In another embodiment, the JNK Inhibitor has the following structure (IIIB): 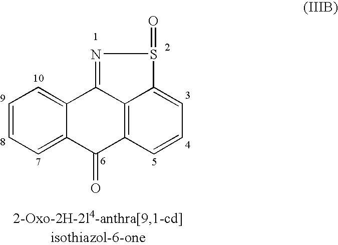
being (i) unsubstituted, (ii) monosubstituted and having a first substituent, or (ii) disubstituted and having a first substituent and a second substituent;
the first or second substituent, when present, is at the 3, 4, 5, 7, 8, 9, or 10 position;
wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b) (c), (d), (e), or (f): 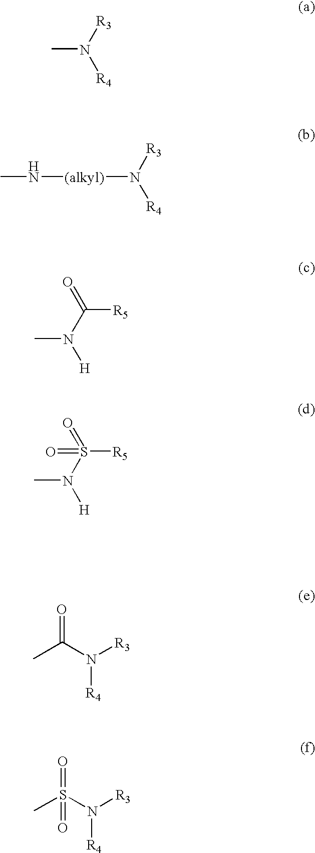
wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and
R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

A subclass of the compounds of structure (IIIB) is that wherein the first or second substituent is present at the 5, 7, or 9 position. In one embodiment, the first or second substituent is present at the 5 or 7 position.

A second subclass of the compounds of structure (IIIB) is that wherein the first or second substituent is independently alkoxy, aryloxy, or a group represented by the structure (a), (c), (d), (e), or (f);
R₃ and R₄ are independently hydrogen, alkyl, cycloalkyl, aryl, arylalkyl, or cycloalkylalkyl; and
R₅ is hydrogen, alkyl, cycloalkyl, aryl, arylalkyl, or cycloalkylalkyl.

In another embodiment, the JNK Inhibitor has the following structure (IIIC): 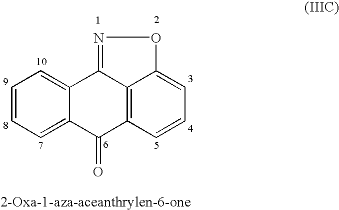
being (i) monosubstituted and having a first substituent or (ii) disubstituted and having a first substituent and a second substituent;
the first or second substituent, when present, is at the 3, 4, 5, 7, 8, 9, or 10 position;
wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c) (d), (e), or (f): 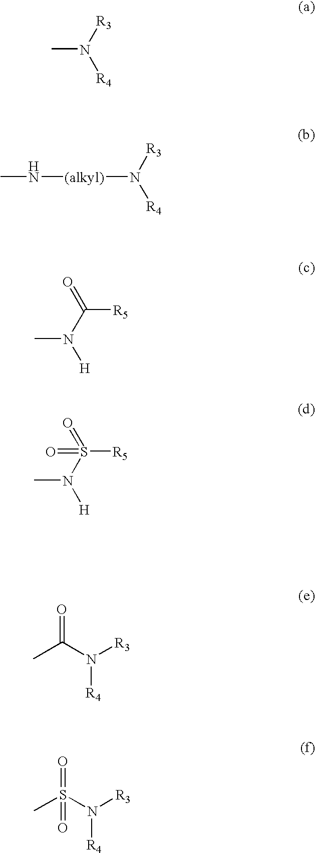
wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and
R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

A subclass of the compounds of structure (IIIC) is that wherein the first or second substituent is present at the 5, 7, or 9 position. In one embodiment, the first or second substituent is present at the 5 or 7 position.

A second subclass of the compounds of structure (IIIC) is that wherein the first or second substituent is independently alkoxy, aryloxy, aminoalkyl, mono-alkylaminoalkyl, di-alkylaminoalkyl, or a group represented by the structure (a), (c), (d), (e), or (f);
R₃ and R₄ are independently hydrogen, alkyl, cycloalkyl, aryl, arylalkyl, or cycloalkylalkyl; and
R₅ is hydrogen, alkyl, cycloalkyl, aryl, arylalkyl, or cycloalkylalkyl.

In another embodiment, the JNK Inhibitor has the following structure (IIID): 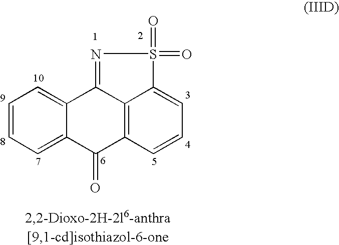
being (i) monosubstituted and having a first substituent present at the 5, 7, or 9 position, (ii) disubstituted and having a first substituent present at the 5 position and a second substituent present at the 7 position, (iii) disubstituted and having a first substituent present at the 5 position and a second substituent present at the 9 position, or (iv) disubstituted and having a first substituent present at the 7 position and a second substituent present at the 9 position;
wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f): 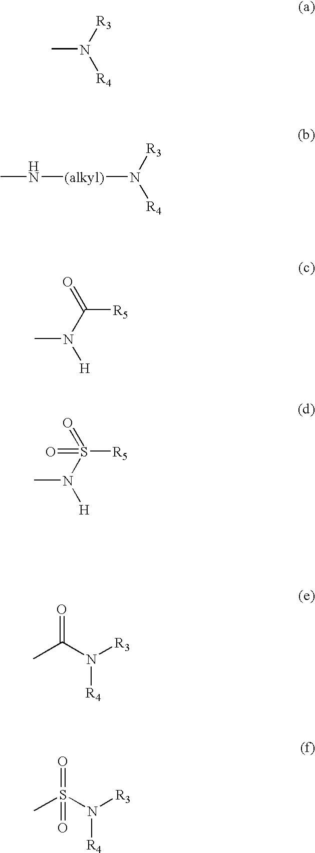
wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and
R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

A subclass of the compounds of structure (IIID) is that wherein the first or second substituent is present at the 5 or 7 position.

A second subclass of the compounds of structure (IIID) is that wherein the first or second substituent is independently alkyl, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (c), (d), (e), or (f).

Another subclass of the compounds of structure (IIID) is that wherein the first and second substituent are independently alkoxy, aryloxy, or a group represented by the structure (a), (c), (d), (e), or (f);
R₃ and R₄ are independently hydrogen, alkyl, cycloalkyl, aryl, arylalkyl, or cycloalkylalkyl; and
R₅ is hydrogen, alkyl, cycloalkyl, aryl, arylalkyl, alkoxycarbonyl, or cycloalkylalkyl.

In another embodiment, the JNK Inhibitor has the following structure (IIIE): 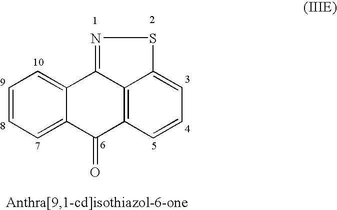
being (i) monosubstituted and having a first substituent present at the 5, 7, or 9 position, (ii) disubstituted and having a first substituent present at the 5 position and a second substituent present at the 9 position, (iii) disubstituted and having a first substituent present at the 7 position and a second substituent present at the 9 position, or (iv) disubstituted and having a first substituent present at the 5 position and a second substituent present at the 7 position;
wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f): 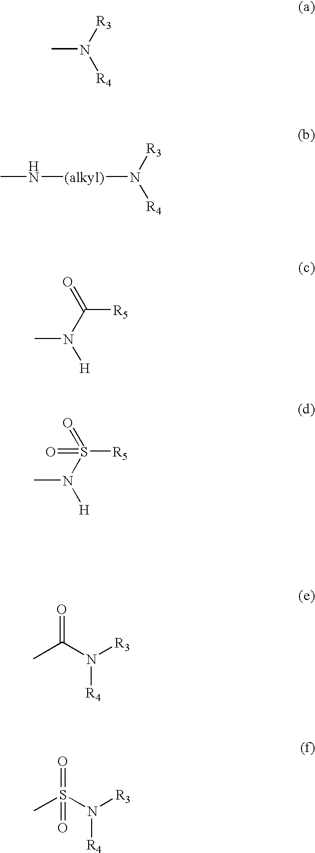
wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

A subclass of the compounds of structure (IIIE) is that wherein the first or second substituent is present at the 5 or 7 position.

A second subclass of the compounds of structure (IIIE) is that wherein the compound of structure (IIIE) is disubstituted and at least one of the substituents is a group represented by the structure (d) or (f).

Another subclass of the compounds of structure (IIIE) is that wherein the compounds are monosubstituted. Yet another subclass of compounds is that wherein the compounds are monosubstituted at the 5 or 7 position with a group represented by the structure (e) or (f).

In another embodiment, the JNK Inhibitor has the following structure (IIIF): 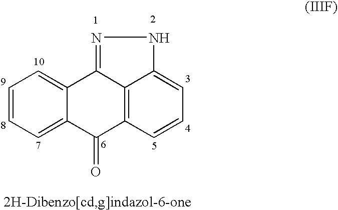
being (i) unsubstituted, (ii) monosubstituted and having a first substituent, or (iii) disubstituted and having a first substituent and a second substituent;
the first or second substituent, when present, is at the 3, 4, 5, 7, 8, 9, or 10 position;
wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono- alkylaminoalkoxy, di-alkylaminoalkoxy, or a group represented by structure (a), (b), (c), (d), (e), or (f): 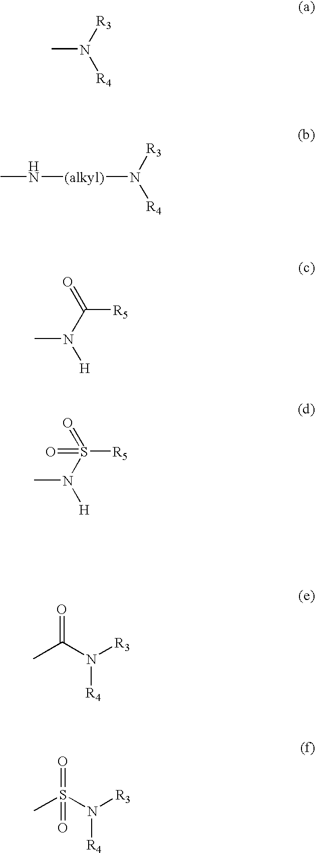
wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and
R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

In one embodiment, the compound of structure (IIIF), or a pharmaceutically acceptable salt thereof is unsubstituted at the 3, 4, 5, 7, 8, 9, or 10 position.

The JNK Inhibitors of structure (III) can be made using organic synthesis techniques known to those skilled in the art, as well as by the methods described in International Publication No. WO 01/12609 (particularly Examples 1-7 at page 24, line 6 to page 49, line 16), published Feb. 22, 2001, as well as International Publication No. WO 02/066450 (particularly compounds AA-HG at pages 59-108), published Aug. 29, 2002, each of which is hereby incorporated by reference in its entirety. Further, specific examples of these compounds can be found in the publications.

Illustrative examples of JNK Inhibitors of structure (III) are: 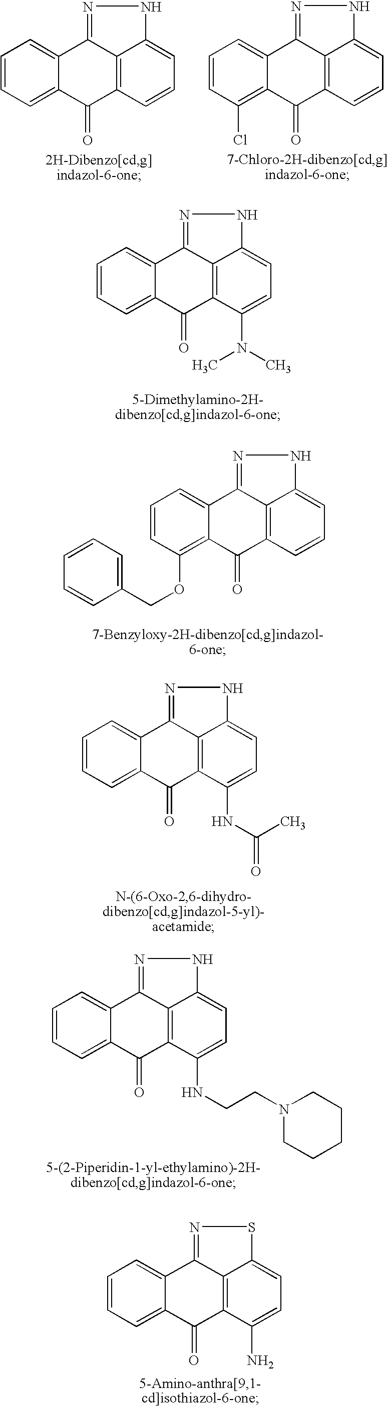 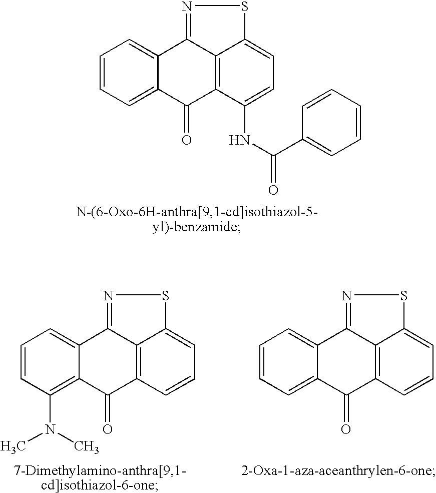
and pharmaceutically acceptable salts thereof.

Other JNK Inhibitors that are useful in the present methods include, but are not limited to, those disclosed in International Publication No. WO 00/39101, (particularly at page 2, line 10 to page 6, line 12); International Publication No. WO 01/14375 (particularly at page 2, line 4 to page 4, line 4); International Publication No. WO 00/56738 (particularly at page 3, line 25 to page 6, line 13); International Publication No. WO 01/27089 (particularly at page 3, line 7 to page 5, line 29); International Publication No. WO 00/12468 (particularly at page 2, line 10 to page 4, line 14); European Patent Publication 1 110 957 (particularly at page 19, line 52 to page 21, line 9); International Publication No. WO 00/75118 (particularly at page 8, line 10 to page 11, line 26); International Publication No. WO 01/12621 (particularly at page 8, line 10 to page 10, line 7); International Publication No. WO 00/64872 (particularly at page 9, line 1 to page, 106, line 2); International Publication No. WO 01/23378 (particularly at page 90, line 1 to page 91, line 11); International Publication No. WO 02/16359 (particularly at page 163, line 1 to page 164, line 25); U.S. Pat. No. 6,288,089 (particularly at column 22, line 25 to column 25, line 35); U.S. Pat. No. 6,307,056 (particularly at column 63, line 29 to column 66, line 12); International Publication No. WO 00/35921 (particularly at page 23, line 5 to page 26, line 14); International Publication No. WO 01/91749 (particularly at page 29, lines 1-22); International Publication No. WO 01/56993 (particularly in at page 43 to page 45); and International Publication No. WO 01/58448 (particularly in at page 39), each of which is incorporated by reference herein in its entirety.

Pharmaceutical compositions including dosage forms of the invention, which comprise an effective amount of a JNK Inhibitor can be used in the methods of the invention. 4.2 Methods for Treating or Preventing Atherosclerosis or Restinosis

The Stent of the Invention can be used to treat or prevent any cardiovascular or renal disease, including atherosclerosis, and in particular, the treatment or prevention of restenosis after vascular intervention such as angioplasty, stent implantation, atherectomy or grafting.

Cardiovascular diseases that the Stent of the Invention are useful for treating or preventing include, but are not limited to, thrombolysis, restenosis, coronary heart disease and myocardial infarction.

Renal diseases that the Stent of the Invention are useful for treating or preventing include, but are not limited to, renal artery stenosis, atherosclerotic ischemic renal disease and fibromuscular dysplasia.

In another embodiment, the Stent of the Invention is useful for treating or preventing a biliary tract carcinoma, esophageal cancer, myocardial infarction, benign prostatic hyperplasia, pancreatic carcinoma, periampullary carcinoma or renal artery stenosis.

In another embodiment, the Stent of the Invention to treat patients having abnormally high levels of circulating macrophage colony stimulating factor.

In another embodiment, the Stent of the Invention is used in combination with vascular intervention including, but not limited to, renal angioplasty, revascularization, percutaneous coronary intervention, percutaneous transluminal coronary angioplasty, carotid percutaneous transluminal angioplasty, coronary by-pass grafting, angioplasty with stent implantation, peripheral percutaneous transluminal intervention of the iliac, femoral or popliteal arteries or surgical intervention using filled artificial grafts.

In one embodiment, the Stent of the Invention is surgically implanted into a patient's artery, vein or capillary. The following table provides a listing of the major systemic arteries into which a Stent of the Invention is implantable:

TABLE I
Major Systemic Arteries

| Artery | Body Areas Supplied |
|---|---|
| Axillary | Shoulder and axilla |
| Brachial | Upper arm |
| Brachiocephalic | Head, neck, and arm |
| Celiac | Divides into left gastric, splenic, and |
| | hepatic arteries |
| Common carotid | Neck |
| Common iliac | Divides into external and internal iliac |
| | arteries |
| Coronary | Heart |
| Deep femoral | Thigh |
| Digital | Fingers |
| Dorsalis pedis | Foot |
| External carotid | Neck and external head regions |
| External iliac | Femoral artery |
| Femoral | Thigh |
| Gastric | Stomach |
| Hepatic | Liver, gallbladder, pancreas, and |
| | duodenum |
| Inferior mesenteric | Descending colon, rectum, and pelvic wall |
| Internal carotid | Neck and internal head regions |
| Internal iliac | Rectum, urinary bladder, external genitalia, |
| | buttocks muscles, uterus and vagina |
| Left gastric | Esophagus and stomach |
| Middle sacral | Sacrum |
| Ovarian | Ovaries |
| Palmar arch | Hand |
| Peroneal | Calf |
| Popliteal | Knee |
| Posterior tibial | Calf |
| Pulmonary | Lungs |
| Radial | Forearm |
| Renal | Kidney |
| Splenic | Stomach, pancreas, and spleen |
| Subclavian | Shoulder |
| Superior mesenteric | Pancreas, small intestine, ascending and |
| | transverse colon |
| Testicular | Testes |
| Ulnar | Forearm |

The optimal dosage of a JNK Inhibitor in a coating for a stent or the material comprising the stent will be readily determined by those skilled in the art and will vary depending on the condition being treated, the particular JNK Inhibitor and mode of administration. Other factors include the weight and condition of the patient. It is to be understood that the present invention has application for both human and veterinary use.

In one embodiment, the Stent of the Invention will comprise about 0.01 mg to about 5000 mg of an effective amount of a JNK Inhibitor. In another embodiment, the Stent of the Invention will comprise about 0.1 mg to about 4500 mg of an effective amount a JNK Inhibitor. In another embodiment, the Stent of the Invention will comprise about 1 mg to about 4000 mg of an effective amount a JNK Inhibitor. In another embodiment, the Stent of the Invention will comprise about 25 mg to about 4000 mg of an effective amount a JNK Inhibitor. In another embodiment, the Stent of the Invention will comprise about 50 mg to about 3000 mg of an effective amount a JNK Inhibitor. In another embodiment, the Stent of the Invention will comprise about 100 mg to about 2000 mg of an effective amount a JNK Inhibitor. In another embodiment, the Stent of the Invention will comprise about 250 mg to about 1500 mg of an effective amount a JNK Inhibitor. In another embodiment, the Stent of the Invention will comprise about 500 mg to about 1000 mg of an effective amount a JNK Inhibitor. In another embodiment, the Stent of the Invention will comprise about 250 mg to about 500 mg of an effective amount a JNK Inhibitor.

Patients who receive stents typically have one or more of the following conditions: abnormal serum lipid levels, hypertension, cigarette smoking, diabetes mellitus, obesity, physical inactivity, hyperhomocysteinemia and chlamydia pneumoniae infection.

In one embodiment, the Stent of the Invention can be implanted into a patient that has previously undergone cardiovascular or renal surgery. In another embodiment, the Stent of the Invention can be implanted into a patient that has not previously undergone cardiovascular or renal surgery. In another embodiment, the Stent of the Invention can be implanted during an endoscopic retrograde cholangiopancreatography (ERCP).

In another embodiment, the Stent of the Invention implanted in a patient prior to undergoing surgery. In one embodiment, the surgery is cardiovascular or renal surgery.

4.3 Stents of the Invention

Examples of stents that can be coated with an effective amount of a JNK Inhibitor or that can comprise a material having an effective amount of a JNK Inhibitor incorporated therein include, but are not limited to, all types of angioplasty devices including a stent or stent graft, a synthetic vascular graft or a biologic vascular graft.

In one embodiment, the stent comprises a polymer. Illustrative polymers include, but are not limited to a polyamide, a polyester, a polystyrene, a polypropylene, a polyacrylate, a polyvinyl, a polycarbonate, a polytetrafluorethylene, a polymethylmethacrylate, a polyethylene, a poly(ethylene terephthalate), a polyalkylene oxalate, a polyurethane, a polysiloxane, a poly(dimethyl siloxane), a polycyanoacrylate, a polyphosphazene, a poly(amino acid), a ethylene glycol I dimethacrylate, a poly(methyl methacrylate), a poly(2-hydroxyethyl methacrylate), a poly(HEMA) or a polyhydroxyalkanoate compound. In one embodiment, the polymer has an effective amount of a JNK Inhibitor incorporated therein. In one embodiment, the polymer is biocompatible.

Illustrative examples of stents include, but are not limited to, esophageal stents, tracheal stents, biliary stents and prostatic stents. The stents can be uncovered, covered or anti-reflux (See U.S. Pat. Nos. 5,984,965 and 5,647,843, each incorporated by reference herein). Any stent, stent graft or tissue engineered vascular graft known in the art can be coated, sealed or filled with a JNK Inhibitor. In one embodiment, the stent is biodegradable (See U.S. Pat. No. 6,423,097, incorporated herein by reference). In another embodiment, the stent is nonbiodegradable. In another embodiment, the stent is self-expanding (See U.S. Pat. No. 6,425,898, incorporated herein by reference). In. another embodiment, the stent is balloon-expandable (See U.S. Pat. No. 5,79,729, incorporated herein by reference). In another embodiment, the stent is made of a hollow tubular wire (See U.S. Pat. No. 5,891,108, incorporated by reference herein).

Specific examples of stents include, but are not limited to, Palmaz, Palmaz-Schatz, Gianturco, Gianturco-Roubin, Gianturco-Rosch, Strecker or memory-shape stents.

In one embodiment, the stent is mounted on a catheter (See U.S. Pat. No. 6,428,570, incorporated herein by reference).

In another embodiment, the stent is combined with a filter device useful for catching any plaques, particles or debris that becomes dislodged during or after implantation of the stent.

In another embodiment, the stent is a fabric-coated metal structure and can be configured into any desired shape or conformation, such as, for example, linear, tapered or bifurcated and may be prepared using fiber technology, such as, e.g., crimped, woven, knitted, velour, double velour, with or without coils.

In another embodiment, the stent is prepared by chemical extrusion, casting or molding using, for example, porous materials, optionally containing an effective amount of a JNK Inhibitor, having linear or random pores that are circular or geometric in shape.

In another embodiment, the stent comprises biomaterial such as decolderized chorioallantoic membranes from the placenta or other collagen material optinally having an effective amount of a JNK Inhibitor incorporated therein.

4.4 Methods For Making a Stent of The Invention

The invention also encompasses methods for making a Stent of the Invention, comprising the step of coating a stent with an effective amount of a JNK Inhibitor. In another embodiment, the coating further comprises a pharmaceutically acceptable carrier. The coating step includes, but is not limited to, dipping, spraying, casting, layering, adding or filling a stent with an effective amount of one or more JNK Inhibitors.

The invention also encompasses methods for making a Stent of the Invention, comprising the step of manufacturing the stent using a material having an effective amount of a JNK Inhibitor incorporated therein. Methods for the manufacture of a stent are well know to those skilled in the art. In another embodiment, the material comprising the Stent of the Invention further comprises a pharmaceutically acceptable carrier. In another embodiment, the material comprising the Stent of the Invention allows for controlled-release of a JNK Inhibitor.

In one embodiment, a stent is coated with an effective amount of a JNK Inhibitor prior to use in the patient. In such an embodiment, the JNK Inhibitor can be coated or sealed on the stent. It should be recognized that multilayer coatings or releaseable coatings are also encompassed. Releaseable coatings can directly deposit a JNK Inhibitor to the area at risk for restenosis.

There are a variety of methods useful for making a Stent of the Invention. The JNK Inhibitor can be applied to the stent by spraying at least one surface of the stent with the JNK Inhibitor in suspension, and allowing the applied surface to dry.

In another embodiment, the stent can be dipped into such a suspension, or a suspension comprising the JNK Inhibitor can be cast over the stent, or by layering a stent with a suspension of the JNK Inhibitor, or the JNK Inhibitor, in solution or suspension form, can be added to a stent, or a stent can be filled with a solution or suspension of the JNK Inhibitor. The JNK Inhibitor can also be applied to the inside surface of a stent. By applying the JNK Inhibitor to the inside of the stent, the JNK Inhibitor can promote proper reendothelialization of the lumen wall, promote wound healing or prevent one or more cardiovascular disease states, such as stenosis, restenosis or intimal and neointimal hyperplasias.

Methods for coating stents are also well-known in the art (e.g., See U.S. Pat. Nos. 6,153,252 and 6,299,604, incorporated by reference herein in their entirety). The preparation of controlled-release coated stents is also well-known in the art (e.g., See U.S. Pat. No. 6,358,556, incorporated by reference herein in its entirety). Other methods of coating stents are well known in the art and are contemplated by the invention (e.g., See U.S. Pat. No. 5,637,113 describes coating stents with a polymer film, U.S. Pat. No. 5,837,313 describes a drug-release stent coating process; both of these patents are incorporated herein in their entireties and for all purposes.

The coating layer(s) should be thin enough so that delivery of the stent by catheter will not be impeded. In one embodiment, the coating is less than about 0.005 inches thick. In another embodiment, the coating is less than about 0.002 inches thick. In another embodiment, the coating is less than about 0.001 inches thick. In another embodiment, the coating is less than about 0.0005 inches thick.

The amount of the JNK Inhibitor to be applied to the stent or incorporated into the stent can be determined empirically by measuring the efficacy of Stents of the Invention having different amounts of the JNK Inhibitor coated thereon or incorporated therein. Also, one skilled in the relevant art is capable of evaluating the efficacy of a Stent of the Invention.

The methods used for implanting the Stent of the Invention, which often involve surgery, are analogous to those used for the implantation of such stents which do not comprise a JNK Inhibitor, and, of course, depend on the nature of the condition to be modified or corrected. The surgery can be performed under either local or systemic anesthesia and, generally, involves an incision, spacing to accommodate the implant, insertion, and suture.

The JNK Inhibitor can be provided as a pharmaceutically acceptable formulation using formulation methods known to those skilled in the art. In addition, the JNK Inhibitor can be incorporated into a biodegradable polymer allowing for sustained release of the compound. Biodegradable polymers and their use are described, for example, in detail in Brem et al., J. Neurosurg. 74:441-446 (1991).

The formulations include those suitable for implantation into a patient. The formulations may be prepared by conventional pharmaceutical techniques. Such techniques include the step of admixing a JNK Inhibitor and a pharmaceutical carrier(s) or excipient(s). In general, the formulations can be prepared by admixing a JNK Inhibitor with liquid carriers or finely divided solid carriers or both, and then, if necessary, shaping the product.

Formulations for coating a stent thus comprise a JNK Inhibitor and optionally a pharmaceutically acceptable carrier, diluent or excipient. In preparing such formulations, the JNK Inhibitor is usually mixed with or diluted by an excipient. When the excipient serves as a diluent, it may be a solid, semi-solid, or liquid material which acts as a vehicle, carrier, or medium for the JNK Inhibitor. Examples of suitable excipients, include but are not limited to lactose, dextrose, sucrose, sorbitol, mannitol, starch, gum acacia, calcium silicate, microcrystalline cellulose, polyvinlypyrrolidinone, cellulose, water, syrup, and methyl cellulose, the formulations can additionally include lubricating agents such as talc, magnesium stearate and mineral oil, wetting agents, emulsifying and suspending agents, preserving agents such as methyl- and propylhydroxybenzoates, sweetening agents or flavoring agents.

The coating or material can be used to provide slow or controlled-release of one or more JNK Inhibitors using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those skilled in the art, including those described herein, can be readily selected for use with the pharmaceutical compositions of the invention.

Controlled-release coatings and material can be designed to initially release an amount of a JNK Inhibitor that promptly produces the desired therapeutic effect, and gradually and continually release other amounts of a JNK Inhibitor to maintain this level of therapeutic effect over an extended period of time. In order to maintain this constant level of JNK Inhibitor in the body, the JNK Inhibitor must be released from the dosage form at a rate that will replace the amount of JNK Inhibitor being metabolized and excreted from the body. Controlled-release of a JNK Inhibitor can be stimulated by various inducers, including, but not limited to, pH, temperature, an enzyme, water, or other physiological conditions or compounds.

4.5 Other Active Agents

The other active agent optionally present in the Stent of the Invention can be any compound that alone or together with a JNK Inhibitor is useful for treating or preventing a cardiovascular or renal disease, including atherosclerosis, and in particular, the treatment or prevention of restenosis after vascular intervention such as angioplasty. For example, the other active agent can be an anticoagulant, such as an RGD peptide-containing compound, heparin, rapamycin, antithrombin compounds, platelet receptor antagonists, an anti-thrombin antibody, an anti-platelet receptor antibody, aspirin, a prostaglandin inhibitor, a platelet inhibitor, or tick anti-platelet peptide. The other active agent can also be a promoter of vascular cell growth, such as a growth factor receptor antagonist, transcriptional activator or translational promoter. Alternatively, the other active agent can be an inhibitor of vascular cell growth, such as a growth factor inhibitor, a growth factor receptor antagonist, a transcriptional repressor or translational repressor, antisense DNA, antisense RNA, a replication inhibitor, an inhibitory antibody, an antibody directed against growth factors, or a bifunctional molecule. The other active agent can also be a cholesterol-lowering agent, a vasodilating agent, or an agent that interferes with an endogenous vasoactive mechanism. Other examples of other active agents include an anti-inflammatory agent, an anti-platelet or fibrinolytic agent, an anti-neoplastic agent, an anti-allergic agent, an anti-rejection agent, an anti-microbial or anti-bacterial or anti-viral agent, a hormone, a vasoactive substance, an anti-invasive factor, an anti-cancer drug, an antibody or lymphokine, an anti-angiogenic agent, a radioactive agent or gene therapy drug. The other active agent can be in its original commercial form, or together with a polymer or protein carrier, to achieve controlled and consistent release.

Illustrative examples of still other active agents include, but are not limited to, IMiDs® and SelCIDs® (Celgene Corporation, New Jersey) (e.g., those disclosed in U.S. Pat. Nos. 6,075,041; 5,877,200; 5,698,579; 5,703,098; 6,429,221; 5,736,570; 5,658,940; 5,728,845; 5,728,844; 6,262,101; 6,020,358; 5,929,117; 6,326,388; 6,281,230; 5,635,517; 5,798,368; 6,395,754; 5,955,476; 6,403,613; 6,380,239; and 6,458,810, each of which is incorporated herein by reference), PDE IV inhibitors (e.g., cilomast, theophylline, zardaverine, rolipram, pentoxyfylline, enoximone), paclitaxel, docetaxel or a derivative thereof, an epothilone, a nitric oxide release agent, heparin, aspirin, coumadin, PPACK, hirudin, polypeptide from angiostatin and endostatin, methotrexate, 5-fluorouracil, estradiol, P-selectin Glycoprotein ligand-1 chimera, abciximab, exochelin, eleutherobin and sarcodictyin, fludarabine, sirolimus, tranilast, VEGF, transforming growth factor (TGF)-beta, Insulin-like growth factor (IGF), platelet derived growth factor (PDGF), fibroblast growth factor (FGF), RGD peptide, a beta or gamma ray emitter (radioactive) agent.

In another embodiment, the Stent of the Invention further comprises an antibiotic agent or an antiviral agent, or mixtures thereof, which can prevent graft rejection.

4.6 Kits

The invention provides a pharmaceutical pack or kit comprising one or more containers containing a Stent of the Invention useful for the treatment or prevention of a cardiovascular or renal disease. Optionally associated with such container(s) can be a notice in the form prescribed by a governmental agency regulating the manufacture, use or sale of pharmaceuticals or biological products, which notice reflects approval by the agency of manufacture, use or sale for human administration; or instructions for the Stent of the Invention's use.

The following examples will serve to further typify the nature of this invention but should not be construed as a limitation in the scope thereof.

### 5. EXAMPLES

5.1 Coating of a Stent

A 5% (w/w) silicone solution in tetrahydrofuran (THF) (HPLC grade, Aldrich or EM Science) is prepared by adding THF and a crosslinker agent into the silicone mixture. A separate 0.5% (w/w) solution of a JNK Inhibitor is prepared. The ratio of W_{drug}/W_{silicone solid} is about 0.1. The coating of the stent in an expanded state is accomplished by spraying one cycle of silicone solution, waiting for a short period of time (about 30 seconds), and spraying one cycle of JNK Inhibitor solution, waiting for a short period of time (about 30 seconds), and then repeating the spraying sequence. The very last spray cycle is silicone solution. For a coating thickness of 30 microns, about 30 cycles each is applied. The number of spray cycles used depends on the solution viscosity, the droplet size and the flow rate. The coated stent is then moved to a convection oven and cured at 150° C. for 45 minutes.

5.2 JNK Inhibitor Activity Assays

The ability of a JNK Inhibitor to inhibit JNK and accordingly, to be useful for the treatment or prevention of a cardiovascular or renal disease, can be demonstrated using one or more of the following assays.

5.2.1 JNK2 Assays

To 10 μL of the JNK Inhibitor in 20% DMSO/80% dilution buffer consisting of 20 mM HEPES (pH 7.6), 0.1 mM EDTA, 2.5 mM magnesium chloride, 0.004% Triton ×100, 2 μg/mL leupeptin, 20 mM β-glycerolphosphate, 0.1 mM sodium vanadate, and 2 mM DTT in water is added 30 μL of 50 ng His6-JNK2 in the same dilution buffer. The mixture is preincubated for 30 minutes at room temperature. Sixty microliters of 10 μg GST-c-Jun(1-79) in assay buffer consisting of 20 mM HEPES (pH 7.6), 50 mM sodium chloride, 0.1 mM EDTA, 24 mM magnesium chloride, 1 mM DTT, 25 mM PNPP, 0.05% Triton ×100, 11 μM ATP, and 0.5 μCi γ-³²P ATP in water is added and the reaction is allowed to proceed for 1 hour at room temperature. The c-Jun phosphorylation is terminated by addition of 150 μL of 12.5% trichloroacetic acid. After 30 minutes, the precipitate is harvested onto a filter plate, diluted with 50 μL of the scintillation fluid and quantified by a counter. The IC₅₀ values are calculated as the concentration of the JNK Inhibitor at which the c-Jun phosphorylation is reduced to 50% of the control value. In one embodiment, JNK Inhibitors have an IC₅₀ value ranging 0.01-10 μM in this assay.

5.2.2 JNK3 Assay

To 10 μL of the JNK Inhibitor in 20% DMSO/80% dilution buffer consisting of 20 mM HEPES (pH 7.6), 0.1 mM EDTA, 2.5 mM magnesium chloride, 0.004% Triton ×100, 2 μg/mL leupeptin, 20 mM β-glycerolphosphate, 0.1 mM sodium vanadate, and 2 mM DTT in water is added 30 μL of 200 ng His6-JNK3 in the same dilution buffer. The mixture is preincubated for 30 minutes at room temperature. Sixty microliter of 10 μg GST-c-Jun(1-79) in assay buffer consisting of 20 mM HEPES (pH 7.6), 50 mM sodium chloride, 0.1 mM EDTA, 24 mM magnesium chloride, 1 mM DTT, 25 mM PNPP, 0.05% Triton ×100, 11 μM ATP, and 0.5 μCi γ-³²P ATP in water is added and the reaction is allowed to proceed for 1 hour at room temperature. The c-Jun phosphorylation is terminated by addition of 150 μL of 12.5% trichloroacetic acid. After 30 minutes, the precipitate is harvested onto a filter plate, diluted with 50 μL of the scintillation fluid and quantified by a counter. The IC₅₀ values are calculated as the concentration of the JNK Inhibitor at which the c-Jun phosphorylation is reduced to 50% of the control value. In one embodiment, JNK Inhibitors have an IC₅₀ value ranging 0.01-10 μM in this assay.

5.2.3 Jurkat T-Cell IL-2 Production Assay

Jurkat T cells (clone E6-1) are purchased from the American Tissue Culture Collection and maintained in growth media consisting of RPMI 1640 medium containing 2 mM L-glutamine (Mediatech), with 10% fetal bovine serum (Hyclone) and penicillin/streptomycin. All cells are cultured at 37° C. in 95% air and 5% CO₂. Cells are plated at a density of 0.2×10⁶ cells per well in 200 μL of media. JNK Inhibitor stock (20 mM) is diluted in growth media and added to each well as a 10× concentrated solution in a volume of 25 μl, mixed, and allowed to pre-incubate with cells for 30 minutes. The vehicle (dimethylsulfoxide) is maintained at a final concentration of 0.5% in all samples. After 30 minutes the cells are activated with PMA (phorbol myristate acetate; final concentration 50 ng/mL) and PHA (phytohemagglutinin; final concentration 2 μg/mL). PMA and PHA are added as a 10× concentrated solution made up in growth media and added in a volume of 25 μL per well. Cell plates are cultured for 10 hours. Cells are pelleted by centrifugation and the media removed and stored at −20° C. Media aliquots are analyzed by sandwich ELISA for the presence of IL-2 as per the manufacturers instructions (Endogen). The IC₅₀ values are calculated as the concentration of the JNK Inhibitor at which the I1-2 production was reduced to 50% of the control value. In one embodiment, JNK Inhibitors have an IC₅₀ value ranging 0.1-30 μM in this assay.

5.2.4 Rat in Vivo Lps-Induced TNF-α Production Assay

Male CD rats procured from Charles River Laboratories at 7 weeks of age are allowed to acclimate for one week prior to use. A lateral tail vein is cannulated percutaneously with a 22-gage over-the-needle catheter under brief isoflurane anesthesia. Rats are administered a JNK Inhibitor either by intravenous injection via the tail vein catheter or oral gavage 15 to 180 min prior to injection of 0.05 mg/kg LPS (E. Coli 055:B5). Catheters are flushed with 2.5 mL/kg of normal injectable saline. Blood is collected via cardiac puncture 90 minutes after LPS challenge. Plasma is prepared using lithium heparin separation tubes and frozen at −80° C. until analyzed. TNF-α levels are determined using a rat specific TNF-α ELISA kit (Busywork). The ED₅₀ values are calculated as the dose of the JNK Inhibitor at which the TNF-α production is reduced to 50% of the control value. In one embodiment, JNK Inhibitors have an ED₅₀ value ranging 1-30 mg/kg in this assay.

5.2.5 Detection of Phosporylated c-Jun

Human umbilical vein endothelial cells (HUVEC) are cultured to 80% confluency and then pre-treated with a JNK Inhibitor (30 μM) at a final concentration of 0.5% DMSO. After 30 minutes, cells are stimulated with TNFα (30 ng/ml) for 20 minutes. Cells are washed, scraped from the plate, lyzed with 2× Laemmli buffer and heated to 100° C. for 5 minutes. Whole cell lysate (approx. 30 μg) is fractionated on Tris-glycine buffered 10% SDS-polyacrylamide gels (Novex, San Diego, Calif.) and transferred to nitrocellulose membrane (Amersham, Piscataway, N.J.). Membranes are blocked with 5% non-fat milk powder (BioRad, Hercules, Calif.) and incubated with antibody to phospho-cJun (1:1000 #91645) (New England Biolabs, Beverly, Mass.) and then donkey anti-rabbit horse radish peroxidase conjugated antibody (1:2500) (Amersham) in phosphate buffered saline with 0.1% Tween-20 and 5% non-fat milk powder. Immunoreactive proteins are detected with chemiluminescence and autoradiography (Amersham). In one embodiment, JNK Inhibitors show greater than 50% inhibition of c-Jun phospborylation at 30 μm in this assay.

Embodiments of the invention described herein are only illustrative of the scope of the invention. A number of references have been cited herein, the entire contents of which have been incorporated by reference herein.

A number of references have been cited, the entire disclosure of which are incorporated herein by reference in their entirety.

1. A stent comprising an effective amount of a JNK Inhibitor.

2. The stent of claim 1 having a coating comprising an effective amount of a JNK Inhibitor.

3. The stent of claim 1 comprising a material having an effective amount of a JNK Inhibitor incorporated therein.

4. The stent according to claim 1, wherein the JNK Inhibitor has the following formula: 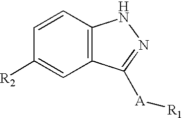
or a pharmaceutically acceptable salt, solvate or stereoisomer thereof,
wherein:
A is a direct bond, —(CH₂)ₐ—, —(CH₂)_{b}CH═CH(CH₂)_{c}—, or —(CH₂)_{b}C≡C(CH₂)_{c}—;
R₁ is aryl, heteroaryl or heterocycle fused to phenyl, each being optionally substituted with one to four substituents independently from R₃;
R₂ is —R₃, —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)_{c}C(═O)R₆, —(CH₂)_{b}NR₅C(═O)R₆, —(CH₂)_{b}NR₅C(=O)NR₆R₇, —(CH₂)_{b}NR₅R₆, —(CH₂)_{b}OR₅,—(CH₂)_{b}SO_{d}R₅ or —(CH₂)_{b}SO₂NR₅R₆;
a is 1, 2, 3, 4, 5 or 6;
b and c are the same or different and at each occurrence independently 0, 1, 2, 3 or 4;
d is at each occurrence 0, 1 or 2;
R₃ is at each occurrence independently halogen, hydroxy, carboxy, alkyl, alkoxy, haloalkyl, acyloxy, thioalkyl, sulfinylalkyl, sulfonylalkyl, hydroxyalkyl, aryl, substituted aryl, arylalkyl, heterocycle, heterocycloalkyl, —C(═O)OR₈, —OC(═O)R₈, —C(═O)NR₈R₉, —C(═O)NR₈OR₉, —SO₂NR₈R₉, —NR₈SO₂R₉, —CN, —NO₂, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₄ is alkyl, aryl, arylalkyl, heterocycle or heterocycloalkyl, each being optionally substituted with one to four substituents independently from R₃, or R₄ is halogen or hydroxy;
R₅, R₆ and R₇ are the same or different and at each occurrence independently hydrogen, alkyl, aryl, arylalkyl, heterocycle or heterocycloalkyl, wherein each of R₅, R₆ and R₇ are optionally substituted with one to four substituents independently from R₃; and R₈ and R₉ are the same or different and at each occurrence independently hydrogen, alkyl, aryl, arylalkyl, heterocycle, or heterocycloalkyl, or R₈ and R₉ taken together with the atom or atoms to which they are bonded form a heterocycle, wherein each of R₈, R₉, and R₈ and R₉ taken together to form a heterocycle are optionally substituted with one to four substituents independently from R₃.

5. The stent according to claim 1, wherein the JNK Inhibitor has the following formula: 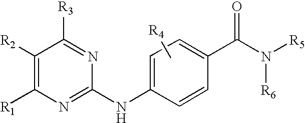
or a pharmaceutically acceptable salt, solvate or stereoisomer thereof,
wherein:
R₁ is aryl or heteroaryl optionally substituted with one to four substituents independently from R₇;
R₂ is hydrogen;
R₃ is hydrogen or lower alkyl;
R₄ represents one to four optional substituents, wherein each substituent is the same or different and independently halogen, hydroxy, lower alkyl or lower alkoxy;
R₅ and R₆ are the same or different and independently —R₈, —(CH₂)ₐC(═O)R₉, —(CH₂)ₐC(═O)OR₉, —(CH₂)ₐC(═O)NR₉R₁₀, —(CH₂)ₐC(═O)NR₉(CH₂)_{b}C(═O)R₁₀, —(CH₂)ₐNR₉C(═O)R₁₀, (CH₂)ₐNR₁₁C(═O)NR₉R₁₀, —(CH₂)ₐNR₉R₁₀, —(CH₂)ₐOR₉, —(CH₂)ₐSO_{c}R₉ or —(CH₂)ₐSO₂NR₉R₁₀;
or R₅ and R₆ taken together with the nitrogen atom to which they are attached to form a heterocycle or substituted heterocycle;
R₇ is at each occurrence independently halogen, hydroxy, cyano, nitro, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(±O)NR₈R₉, —C(═O)NR₈OR₉, —SO_{c}R₈, —SO_{c}NR₈R₉, —NR₈SO_{c}R₉, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₈, R₉, R₁₀ and R₁₁ are the same or different and at each occurrence independently hydrogen, alkyl, substituted alkyl, aryl, arylalkyl, heterocycle or heterocycloalkyl;
or R₈ and R₉ taken together with the atom or atoms to which they are attached to form a heterocycle;
a and b are the same or different and at each occurrence independently 0, 1, 2, 3 or 4; and
c is at each occurrence 0, 1 or 2.

6. The stent according to claim 1, wherein the JNK Inhibitor has the following formula: 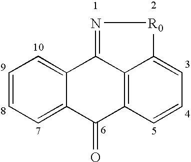
or a pharmaceutically acceptable salt, solvate or stereoisomer thereof,
wherein R₀ is —O—, —S—, —S(O)—, —S(O)₂—, NH or —CH₂—;
the compound being (i) unsubstituted, (ii) monosubstituted and having a first substituent, or (iii) disubstituted and having a first substituent and a second substituent;
the first or second substituent, when present, is at the 3, 4, 5, 7, 8, 9, or 10 position, wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by formula (a), (b), (c), (d), (e), or (f): 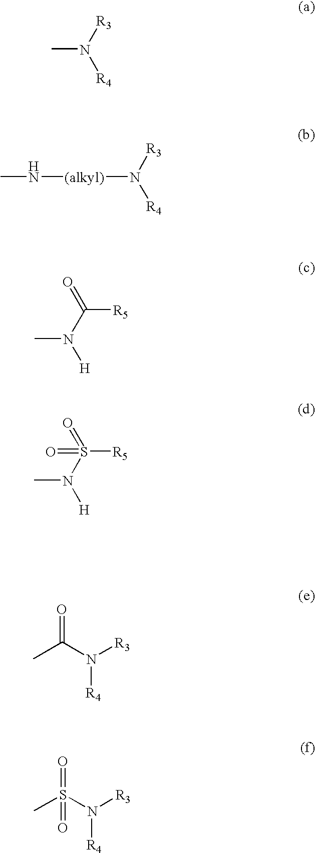
wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and
R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, mono-alkylaminoalkyl, or di-alkylaminoalkyl.

7. The stent according to claim 2 wherein the coating comprises a pharmaceutically acceptable carrier.

8. The stent according to claim 1 wherein the stent is a stent graft.

9. The stent according to claim 1 wherein the stent comprises a polymer.

10. The stent according to claim 9 in which the polymer is a polyamide, a polyester, a polystyrene, a polypropylene, a polyacrylate, a polyvinyl, a polycarbonate, a polytetrafluorethylene, a polymethylmethacrylate, a polyethylene, a poly(ethylene terephthalate), a polyalkylene oxalate, a polyurethane, a polysiloxane, a poly(dimethyl siloxane), a polycyanoacrylate, a polyphosphazene, a poly(amino acid), a ethylene glycol I dimethacrylate, a poly(methyl methacrylate), a poly(2-hydroxyethyl methacrylate), a poly(HEMA),or a polyhydroxyalkanoate compound.

11. The stent according to claim 2 wherein the coating is a controlled-release coating.

12. A method for making the stent of claim 2, comprising the step of coating a stent with an effective amount of a JNK Inhibitor.

13. The method according to claim 12 wherein the stent is a stent graft.

14. The stent according to claim 3 wherein the material having an effective amount of a JNK Inhibitor incorporated therein allows for controlled-release of the JNK Inhibitor.

15. A method for making the stent of claim 3, comprising manufacturing a stent with material having an effective amount of a JNK Inhibitor incorporated therein.

16. A method for treating or preventing a cardiovascular or renal disease in a patient, comprising implanting the stent of claim 1 into a patient in need thereof.

17. A method for treating or preventing atherosclerosis in a patient, comprising implanting the stent of claim 1 into a patient in need thereof.

18. The method of claim 16 further comprising surgical intervention.

19. The method of claim 17 further comprising surgical intervention.

20. The method of claim 18 wherein the surgical intervention involves percutaneous coronary intervention, revascularization, percutaneous transluminal coronary angioplasty, carotid percutaneous transluminal angioplasty coronary by-pass grafting or coronary angioplasty with stent implantation.

21. The method of claim 18 wherein the surgical intervention involves renal angioplasty; peripheral percutaneous transluminal intervention of the iliac, femoral or popliteal arteries; or surgical intervention using impregnated artificial grafts.

22. The method of claim 16 wherein the stent is a stent graft.

23. The method of claim 17 wherein the stent is a stent graft.

24. The method of claim 20 wherein the implanting occurs prior to the administration of angioplasty.

25. The method of claim 20 wherein the implanting occurs during the administration of angioplasty.

26. The method of claim 20 wherein the implanting occurs after the administration of angioplasty.

27. A kit comprising the stent of claim 1 and directions for its use.
